(19) United States
(12) Patent Application Publication
(10) Pub. No.: US 2024/0254642 A1
(43) Pub. Date: Aug. 1, 2024

(54) MANUFACTURING AND USE OF CO-DOPED MULTI-METALLIC ELECTROCATALYSTS FOR UPGRADING OF CO TO PROPANOL

(71) Applicant: THE GOVERNING COUNCIL OF THE UNIVERSITY OF TORONTO, Toronto (CA)

(72) Inventors: Xue WANG, Toronto (CA); Edward SARGENT, Toronto (CA)

(73) Assignee: THE GOVERNING COUNCIL OF THE UNIVERSITY OF TORONTO, Toronto, ON (CA)

(21) Appl. No.: 18/564,001

(22) PCT Filed: May 25, 2022
(86) PCT No.: PCT/CA2022/050828

§ 371 Date: Nov. 24, 2023

Related U.S. Application Data
(60) Provisional application No. 63/192,842, filed on May 25, 2021.

(51) Int. Cl. C25B 11/097; C07C 29/48; C25B 3/07; C25B 3/25; C25B 3/26; C25B 11/032

(52) U.S. Cl. CPC C25B 11/097; C07C 29/48; C25B 3/07; C25B 3/25; C25B 3/26; C25B 11/032

(57) ABSTRACT

The present disclosure relates to the manufacturing and use of co-doped multi-metallic electrocatalysts for electroreduction of CO or CO₂ to produce n-propanol. The co-doped multi-metallic electrocatalyst includes Cu as well as Ag and a secondary dopant, such as Ruthenium (Ru), Rhodium (Rh), Iridium (Ir), Palladium (Pd), Gold (Au) or Platinum (Pt). The co-doped multi-metallic electrocatalyst can be manufactured using a two-stage method where Cu nanoparticles are first doped with Ru and then doped with Ag. The co-doped multi-metallic electrocatalysts facilitate adsorption of CO, C1-C1 coupling, C1-C2 coupling and certain kinetics for the production of propanol by electroreduction with good selectivity at high current densities.

### TECHNICAL FIELD

The technical field generally relates to catalytic methods for monoxide (CO) and carbon dioxide (CO₂) reduction, and more particularly to electrocatalysts composed of metallic materials, associated methods of manufacture and use in electrochemical reduction for the production of C3 products such as propanol.

### BACKGROUND

The electrosynthesis of C3 products from carbon monoxide (CO) and carbon dioxide (CO₂) addresses certain needs for long-term storage of renewable electricity. However, current performance remains below targets for practical applications. There is a need for improvements for efficient electrocatalytic reduction of gases, such as CO and CO₂, for producing C3 products.

### SUMMARY

Various aspects of the technology are described herein and relate to the manufacturing and use of electrocatalysts for converting CO or CO₂ to produce products, such as n-propanol, where the electrocatalyst includes a multi-metallic material comprising copper (Cu) that is co-doped with Ag and a secondary dopant metal such as Ruthenium (Ru), Rhodium (Rh), Palladium (Pd), Platinum (Pt), Gold (Au) and/or Iridium (Ir). The co-doped multi-metallic material can be made using a two-stage manufacturing method where the Cu material is first doped with the secondary dopant metal followed by doping with silver, which can be performed using galvanic replacement. Embodiments of the co-doped multi-metallic material were found to provide enhanced performance for the production of C3 products, such as propanol, in electrochemical cells operated at high current densities.

In some implementations, there is provided a method of manufacturing a co-doped multi-metallic electrocatalyst for use in electroreduction, the method comprising: providing a copper (Cu) material comprising Cu nanoparticles; in a first doping stage, doping the Cu material with a first-stage dopant metal selected from Ruthenium (Ru), Rhodium (Rh), Iridium (Ir), Palladium (Pd), Gold (Au) and Platinum (Pt) to produce a doped Cu material; and in a second doping stage, doping the doped Cu material with silver (Ag) to produce the co-doped multi-metallic material.

In some implementations, the first doping stage comprises first-stage galvanic replacement of Cu atoms with atoms of the first-stage dopant metal; and optionally the first-stage galvanic replacement comprises contacting the Cu material with a first-stage doping solution comprising cations of the first-stage dopant metal. The first-stage doping solution can include a chloride salt of the first-stage dopant metal or a nitrate salt of the first-stage dopant metal.

In some implementations, the second doping stage comprises second-stage galvanic replacement of Cu atoms with Ag atoms; and optionally the second-stage galvanic replacement comprises contacting the doped Cu material with a second-stage doping solution comprising Ag cations. The second-stage doping solution can include AgNO₃.

In some implementations, the co-doped multi-metallic material is a tri-metallic material. Optionally, the co-doped multi-metallic material has a first-stage dopant concentration between 0.5 wt % and 10 wt %, or between 1 wt % and 3 wt %, measured with XPS. The co-doped multi-metallic material can optionally have a Ag concentration between 1 wt % and 10 wt %, or between 3 wt % and 5 wt %, measured with XPS. Optionally, the co-doped multi-metallic material has a first-stage dopant to Ag ratio between 1:1 and 1:10, or between 1:2 and 1:7, or between 1:3 and 1:5, measured with XPS.

In some implementations, the Cu nanoparticles are deposited onto a gas diffusion substrate prior to the first and second doping stages. Optionally, the Cu nanoparticles are deposited in a Cu layer on a side of the gas diffusion substrate, the Cu layer has a thickness between 30 microns and 100 microns, and the co-doped multi-metallic material has a morphology that is the same as that of the Cu nanoparticles with the morphology optionally being generally spheroid in shape, determined from SEM or TM imaging. The co-doped multi-metallic material can be in the form of nanoparticles. The nanoparticles of the co-doped multi-metallic material can have an average size between about 20 nm and about 200 nm, or between about 70 nm and about 150 nm, measured based on SEM or TEM imaging.

In some implementations, the method also includes: depositing the Cu nanoparticles onto a substrate to form a coated substrate; immersing the coated substrate in a first-stage doping solution comprising the first-stage dopant metal in cationic form to induce galvanic replacement and form a first-stage coated substrate comprising the doped Cu material; removing the first-stage coated substrate from the first-stage doping solution; immersing the first-stage coated substrate in a second-stage doping solution comprising Ag in cationic form to induce galvanic replacement and form a second-stage coated substrate comprising the co-doped multi-metallic material; and removing the second-stage coated substrate from the second-stage doping solution.

In some implementations, the first-stage doping solution has a first-stage dopant metal concentration between 1 micromole/L and 10 millimole/L, and the first-stage doping solution has a temperature between 25 degrees Celsius and 80 degrees Celsius. The method can include, after removing first-stage coated substrate from the first-stage doping solution, washing the coated substrate from the first-stage doping solution with deionized water; as well as drying the washed coated substrate with an inert gas. In some implementations, the second-stage doping solution has a second-stage dopant metal concentration between 1 micromole/L and 10 millimole/L, the second-stage doping solution has a second-stage temperature between 25 degrees Celsius and 80 degree Celsius, and the method also includes washing the second-stage coated substrate with deionized water and drying the washed second-stage coated substrate with an second-stage inert gas, such as N₂.

In some implementations, there is provided a use of a co-doped multi-metallic electrocatalyst for electroreduction of CO or CO₂ to produce n-propanol, the co-doped multi-metallic electrocatalyst comprising copper (Cu) co-doped with silver (Ag) and a secondary dopant selected from Ruthenium (Ru), Rhodium (Rh), Iridium (Ir), Palladium (Pd), Gold (Au) and Platinum (Pt).

In some implementations, there is provided a process for electrochemical production of propanol from a carbon-containing gas selected from CO and CO₂, comprising: contacting the carbon-containing gas and an electrolyte with an electrode comprising the co-doped multi-metallic electrocatalyst as manufactured by the method as defined herein or as defined otherwise herein, such that the carbon-containing gas contacts the electrocatalyst; applying a voltage to provide a current density to cause the carbon-containing gas contacting the electrocatalyst to be electrochemically converted into propanol; and recovering the propanol.

In some implementations, there is provided a system for electroreduction of a carbon-containing gas selected from CO and CO₂ to produce propanol, comprising: an electrolytic cell configured to receive a liquid electrolyte and the carbon-containing gas; an anode; a cathode comprising an electrocatalyst as manufactured by the method described herein or as defined otherwise herein; and a voltage source to provide a current density to cause the CO and/or CO₂ gas contacting the electrocatalyst to be electrochemically converted into propanol.

In some implementations, there is provided a co-doped multi-metallic electrocatalyst for electroreduction of CO or CO₂ to produce n-propanol, comprising copper (Cu) co-doped with silver (Ag) and a secondary dopant selected from Ruthenium (Ru), Rhodium (Rh), Iridium (Ir), Palladium (Pd), Gold (Au) and Platinum (Pt), wherein the co-doped multi-metallic electrocatalyst has a secondary dopant concentration between 0.5 wt % and 10 wt % measured with XPS, a Ag concentration between 1 wt % and 10 wt % measured with XPS, and a secondary dopant to Ag ratio between 1:1 and 1:10 measured with XPS.

In some implementations, there is provided a co-doped multi-metallic electrocatalyst for electroreduction of CO or CO₂ to produce n-propanol, comprising copper (Cu) co-doped with silver (Ag) and a secondary dopant, wherein the co-doped multi-metallic electrocatalyst has a secondary dopant concentration to achieve an increase in electroreduction performance compared to both Cu-only catalyst and Ag-only-doped Cu catalyst at a current density above 100 mA cm⁻².

The co-doped multi-metallic electrocatalyst, processes, methods, systems and/or uses can further include one or more features of the electrocatalyst, processes, systems and/or uses as described herein.

### BRIEF DESCRIPTION OF DRAWINGS

FIG. 1. DFT calculations on C₁-C₁ and C₁-C₂ coupling. a, The calculated activation energy (Eₐ) for C₁-C₁ and C₁-C₂ coupling on screened Ag—X—Cu (X=Au, Pd, Pt, Ni, Fe, and Ru; X is categorized into three different groups according to the CO adsorption energies with the reference to Cu, i.e., weak: Au; intermediate: Pd, Pt, and Ni; strong: Fe and Ru). The Eₐ (C₁-C₁ coupling) and Eₐ (C₁-C₂ coupling) on Ag—Cu and Cu catalyst systems are also calculated for comparison. b, Reaction coordinate diagram for C₁-C₁ and C₁-C₂ coupling on Ag—Ru—Cu, Ag—Cu, and Cu catalyst systems. TS1 and TS2 denote the transition state of C₁-C₁ and C₁-C₂ coupling, i.e., *CO—*CO and *CO—*OCCO, respectively.

FIG. 2. Structural and compositional analyses of the Ag—Ru—Cu catalyst. (a to c) Bright field scanning transmission electron microscopy (BF-STEM) image (a), high-angle annular dark-field STEM (HAADF-STEM) image (b) of Ag—Ru—Cu catalyst and the corresponding of EDS elemental mapping of Cu, Ag, and Ru (c). (d to f) High-resolution XPS spectra of Cu 2p (d), Ag 3d (e), and Ru 3p_{3/2} (f) for the Ag—Ru—Cu catalyst.

FIG. 3. CORR performance of different cathode electrodes. a, C₂₊ product distribution under different current densities for Ag—Ru—Cu and Cu electrodes. Error bars represent the standard deviation (SD) of three independent samples. Data are presented as mean values+/−SD. b, Partial current densities of C₂₊ products for Ag—Ru—Cu and Cu electrodes under different potentials. Error bars represent the standard deviation of potentials (>660 data points collected in one experiment) during the constant-current electrolysis. Data are presented as mean values+/−SD. c, n-Propanol (n-PrOH) FEs and partial n-propanol current densities on different electrodes at various current densities. Error bars represent the standard deviation of three independent samples. Data are presented as mean values+/−SD. d, Comparison of FE_{n-PrOH}/FE_{C}_{₂} ratios on different electrodes at various current densities. e, Liquid product distribution and cell voltage during 102 h operation of CORR at a constant current of 1.5 A. CO feed rate in all these experiments is 47.0 mL min⁻¹.

FIG. 4. In situ characterization and n-propanol electrosynthesis in a larger electrolyzer. a, In situ Raman spectra of different catalysts under different applied potentials vs. the reversible hydrogen electrode (RHE) using 1 M KOH electrolyte during CORR. The regions of 250-450 cm⁻¹, 1900-2000 cm⁻¹, and 2000-2150 cm⁻¹ are marked by orange, blue, and yellow, respectively. b, FEs toward n-propanol and C₂₊ products as well as SPCC with different CO feed rates at the applied current of 4.5 A in the A=15 cm² MEA electrolyzer.

FIG. 5. Breakdown of the plant-gate levelized cost per tonne of n-propanol and the corresponding quantity of ethanol, ethylene, and H₂ produced on Ag—Ru—Cu at the current density of 300 mA cm⁻². The TEA findings are calculated based on the CORR performance in the A=15 cm² MEA electrolyzer with the CO feed rate of 20.7 mL min⁻¹.

FIG. 6. Geometries of initial, transition, and final state of C₁-C₁ and C₁-C₂ coupling on Cu surface. a-f, Side views of initial, transition, and final state, including 2*CO (a), *CO—*CO (b), *OCCO (c), *CO+*OCCO (d), *CO—*OCCO (e), and *OCCOCO (f). g-l, Top views of initial, transition, and final state, including 2*CO (g), *CO—*CO (h), *OCCO (i), *CO+*OCCO (j), *CO—*OCCO (k), and *OCCOCO (l). Red, grey, and orange balls stand for oxygen, carbon, and copper atoms, respectively. Water molecules are shown as lines. 2*CO, *CO—*CO, and *OCCO denote the initial, transition, and final state of C₁-C₁ coupling. *CO+*OCCO, *CO—*OCCO, and *OCCOCO denote the initial, transition, and final state of C₁-C₂ coupling. These notations are used throughout the Supplementary Information.

FIG. 7. Geometries of initial, transition, and final state of C₁-C₁ and C₁-C₂ coupling on Ag—Cu surface. a-f, Side views of initial, transition, and final state, including 2*CO (a), *CO—*CO (b), *OCCO (c), *CO+*OCCO (d), *CO—*OCCO (e), and *OCCOCO (f). g-l, Top views of initial, transition, and final state, including 2*CO (g), *CO—*CO (h), *OCCO (i), *CO+*OCCO (j), *CO—*OCCO (k), and *OCCOCO (l). Blue balls stand for silver atoms. This notation is used throughout the Supplementary Information.

FIG. 8. Geometries of initial, transition, and final state of C₁-C₁ and C₁-C₂ coupling on Ag—Au—Cu surface. a-f, Side views of initial, transition, and final state, including 2*CO (a), *CO—*CO (b), *OCCO (c), *CO+*OCCO (d), *CO—*OCCO (e), and *OCCOCO (f). g-l, Top views of initial, transition, and final state, including 2*CO (g), *CO—*CO (h), *OCCO (i), *CO+*OCCO (j), *CO—*OCCO (k), and *OCCOCO (l). Yellow balls stand for gold atoms. This notation is used throughout the Supplementary Information.

FIG. 9. Geometries of initial, transition, and final state of C₁-C₁ and C₁-C₂ coupling on Ag—Pd—Cu surface with adsorbed *CO near the coupling sites. a-f, Side views of initial, transition, and final state, including 2*CO (a), *CO—*CO (b), *OCCO (c), *CO+*OCCO (d), *CO—*OCCO (e), and *OCCOCO (f). g-l, Top views of initial, transition, and final state, including 2*CO (g), *CO—*CO (h), *OCCO (i), *CO+*OCCO (j), *CO—*OCCO (k), and *OCCOCO (l). Brown balls stand for palladium atoms. This notation is used throughout the Supplementary Information.

FIG. 10. Geometries of initial, transition, and final state of C1-C1 and C1-C2 coupling on Ag—Pt—Cu surface with adsorbed *CO near the coupling sites. a-f, Side views of initial, transition, and final state, including 2*CO (a), *CO—*CO (b), *OCCO (c), *CO+*OCCO (d), *CO—*OCCO (e), and *OCCOCO (f). g-l, Top views of initial, transition, and final state, including 2*CO (g), *CO—*CO (h), *OCCO (i), *CO+*OCCO (j), *CO—*OCCO (k), and *OCCOCO (l). Grey balls stand for platinum atoms. This notation is used throughout the Supplementary Information.

FIG. 11. Geometries of initial, transition, and final state of C₁-C₁ and C₁-C₂ coupling on Ag—Ni—Cu surface with adsorbed *CO near the coupling sites. a-f, Side views of initial, transition, and final state, including 2*CO (a), *CO—*CO (b), *OCCO (c), *CO+*OCCO (d), *CO—*OCCO (e), and *OCCOCO (f). g-l, Top views of initial, transition, and final state, including 2*CO (g), *CO—*CO (h), *OCCO (i), *CO+*OCCO (j), *CO—*OCCO (k), and *OCCOCO (l). Indigo balls stand for nickel atoms. This notation is used throughout the Supplementary Information.

FIG. 12. Geometries of initial, transition, and final state of C₁-C₁ and C₁-C₂ coupling on Ag—Fe—Cu surface with adsorbed *CO near the coupling sites. a-f, Side views of initial, transition, and final state, including 2*CO (a), *CO—*CO (b), *OCCO (c), *CO+*OCCO (d), *CO—*OCCO (e), and *OCCOCO (f). g-l, Top views of initial, transition, and final state, including 2*CO (g), *CO—*CO (h), *OCCO (i), *CO+*OCCO (j), *CO—*OCCO (k), and *OCCOCO (l). Purple balls stand for iron atoms. This notation is used throughout the Supplementary Information.

FIG. 13. Geometries of initial, transition, and final state of C1-C1 and C1-C2 coupling on Ag—Ru—Cu surface with adsorbed *CO near the coupling sites. a-f, Side views of initial, transition, and final state, including 2*CO (a), *CO—*CO (b), *OCCO (c), *CO+*OCCO (d), *CO—*OCCO (e), and *OCCOCO (f). g-l, Top views of initial, transition, and final state, including 2*CO (g), *CO—*CO (h), *OCCO (i), *CO+*OCCO (j), *CO—*OCCO (k), and *OCCOCO (l). Green balls stand for ruthenium atoms. This notation is used throughout the Supplementary Information.

FIG. 14. DFT calculations on Ru doping site and *CO adsorption sequence. a, Surface atom index for determining the Ru doping site, i.e., blue squares denote the 1st neighboring Cu atom around the doped-Ag atom. Grey and orange circles denote the 2nd neighboring Cu atom around the doped-Ag atom. b-d, Geometries and the corresponding energies for different Ru doping sites, 1st neighboring (b), 2nd neighboring marked by the grey circle (c), 2nd neighboring marked by the orange circle (d). e-h, Geometries and the corresponding adsorption energies of the most favorable configurations after the 1 st *CO (6_RuTop) (e), the 2nd *CO (236_Cu2RuFccHollow+679_Cu2RuHcpHollow) (f), the 3rd *CO (236_Cu2RuFccHollow+467_Cu2RuFccHollow+569_Cu2RuFccHollow) (g), and the 4th *CO (256_Cu2RuHcpHollow+346_Cu2RuHcpHollow+679_Cu2RuHcpHollow+8_CuTop) (h) adsorbed on the Ag—Ru—Cu surface. The calculated *CO adsorption energies on different sites are summarized in table S3.

FIG. 15. Comparison of the average adsorption energies of *CO (Ē_{*CO}) and adsorption energies of the reaction intermediate *OCCO (E_{*OCCO}) on Ag—Ru—Cu, Ag—Cu, and Cu catalyst systems.

FIG. 16. SEM images of catalysts. a, Cu. b, Ag—Ru—Cu. The SEM measurements were repeated at least twice independently with similar results.

FIG. 17. XRD patterns for different electrodes and the PTFE substrate. The peaks marked by gray dash lines come from PTFE substrates.

FIG. 18. In-depth elemental profile of the Ag—Ru—Cu electrode via sputtering XPS. In-depth XPS analyses suggest that the galvanic replacement occurred mainly near the electrode surface, within a depth of ˜50 nm.

FIG. 19. Photograph showing the MEA electrolyzer with a 5 cm2 active geometric area of the flow filed on each side.

FIG. 20. Schematic diagram of the MEA system.

FIG. 21. NMR spectra of liquid products. a, Representative 1H-NMR spectrum of liquid products collected from the cathode side. b, Representative 1H-NMR spectrum of liquid products collected from the anolyte. The peaks near 2.21 ppm come from acetone which is used to wash NMR tubes.

FIG. 22. SEM images of Ag—Cu catalysts. The SEM measurements were repeated at least twice independently with similar results.

FIG. 23. Structural and compositional analyses of the Ag—Cu catalysts. a, BF-STEM image of Ag—Cu nanoparticles. b, HAADF-STEM image and the corresponding EDX elemental mapping of Cu and Ag for Ag—Cu nanoparticles. The TEM measurements were repeated at least twice independently with similar results.

FIG. 24. XPS analysis the Ag—Cu catalysts. a,b, High-resolution XPS spectra of Cu 2p (a) and Ag 3d (b) for the prepared Ag—Cu catalysts.

FIG. 25. Structural and compositional analyses of the Ag—Au—Cu catalysts. a, SEM images of Ag—Au—Cu catalysts. The SEM measurements were repeated at least twice independently with similar results. b,c, HAADF-STEM image (b) of Ag—Au—Cu catalyst and the corresponding of EDS elemental mapping of Cu, Au, and Ag (c). The TEM measurements were repeated at least twice independently with similar results.

FIG. 26. XPS analysis the Ag—Au—Cu catalysts. a-c, High-resolution XPS spectra of Cu 2p (a), Ag 3d (b), and Au 4f (c) for the Ag—Au—Cu catalysts.

FIG. 27. Structural and compositional analyses of the Ag—Pd—Cu catalysts. a, SEM images of Ag—Pd—Cu catalysts. The SEM measurements were repeated at least twice independently with similar results. b,c, HAADF-STEM image (b) of Ag—Pd—Cu catalyst and the corresponding of EDS elemental mapping of Cu, Pd, and Ag (c). The TEM measurements were repeated at least twice independently with similar results.

FIG. 28. XPS analysis the Ag—Pd—Cu catalysts. a-c, High-resolution XPS spectra of Cu 2p (a), Ag 3d (b), and Pd 3d (c) for the Ag—Pd—Cu catalysts.

FIG. 29. CORR performance of Ag—Au—Cu electrodes. a, Product distribution under different current densities for Ag—Au—Cu electrodes. b, Comparison of n-PrOH FEs on different electrodes at various current densities. CO feed rate in all these experiments is 47.0 mL cm⁻².

FIG. 30. CORR performance of Ag—Pd—Cu electrodes. a, Product distribution under different current densities for Ag—Pd—Cu electrodes. b, Comparison of n-PrOH FEs on different electrodes at various current densities. CO feed rate in all these experiments is 47.0 mL cm⁻².

FIG. 31. Product distribution during 102 hours of CORR test under the constant current of 1.5 A.

FIG. 32. Structural and compositional analyses of the Ag—Ru—Cu catalysts after stability test. a, Secondary electron image and b-f, the corresponding of EDX elemental mapping of Cu (b), Ag (c), Ru (d), and K (e) for Ag—Ru—Cu catalysts after stability test. The elemental K is derived from the adsorption of K+ from electrolyte on catalysts during the test. Both SEM and TEM measurements were repeated at least twice independently with similar results.

FIG. 33. XPS analysis the Ag—Ru—Cu catalysts after stability test. a, High-resolution Cu 2p spectrum of Ag—Ru—Cu catalysts after stability test. b, High-resolution Ag 3d spectrum of Ag—Ru—Cu catalysts after stability test. In the binding energy region of 370-384 eV, we also observe the signal of K 2s which comes from the adsorption of K+ from electrolyte on the catalysts during the CORR performance test. c, High-resolution Ru 3p3/2 spectrum of Ag—Ru—Cu catalysts after stability test.

FIG. 34. In situ Cu K-edge XANES spectra of different catalysts during CORR by applying 300 mA cm⁻². Bulk Cu foil, CuO, and Cu₂O are shown as references.

FIG. 35. Schematic diagram of the electrochemical cell for in situ Raman measurement.

FIG. 36. In situ Raman spectra of Ag—Ru—Cu in Ar-saturated 1 M KOH electrolyte under different applied potentials. Ar was continuously supplied to the gas chamber during the measurement.

FIG. 37. Photograph showing the MEA electrolyzer with a 15 cm² active geometric area of the flow filed on each side.

FIG. 38. A representative of chronopotentiometric curve measured for the scaled MEA electrolyzer operated at 4.5 A. The active geometric areas of the cathode and anode electrodes are 15 cm²; CO feed rate is 20.7 mL min⁻¹.

FIG. 39. Model used to calculate the plant-gate levelized cost for TEA.

FIG. 40. Sensitivity analysis of the plant-gate levelized cost per tonne of n-propanol and the corresponding quantity of ethanol, ethylene, and H₂. The parameters in the better, base, and worse cases are listed in table S7. All the other parameters used in calculations are same as those used in the calculation listed in Supplementary Text.

FIG. 41. TEA of n-propanol electrosynthesis showing the plant-gate levelized cost per tonne of n-propanol and the corresponding quantity of ethanol, ethylene, and H₂ as function of SPCC and current density. All the other parameters used in calculations are the same as those used in the calculation listed in Supplementary Text.

### DETAILED DESCRIPTION

The present description relates to the manufacturing and use of co-doped multi-metallic electrocatalysts for the reduction of CO or CO₂ to produce C3 products, such as propanol. The electrocatalysts can be manufactured using a two-stage method to incorporate the different dopant metals into Cu in distinct stages, and the electrocatalyst can be used for the electroreduction of CO or CO₂ while providing enhanced performance compared other catalysts.

More particularly, the co-doped multi-metallic electrocatalysts can be manufactured using a two-stage method where Cu nanoparticles are first doped with Ru, Rh, Ir, Pt, Au or Pd and then doped with Ag. The Cu nanoparticles can be deposited on a substrate and then the sequential doping stages can be performed by galvanic replacement. The substrate can be a gas diffusion membrane that can be used as part of the cathode in an electrochemical cell for conversion of CO into propanol at high current densities. For example, it was found that silver-ruthenium (Ag—Ru) co-doped copper (Cu) electrocatalysts provided enhanced performance compared to corresponding electrocatalysts composed of Cu only or Cu doped only with Ag. In some embodiments, the co-doped multi-metallic electrocatalysts were found to achieve a record n-propanol Faradaic efficiency of (37±3) % at a production rate of (111±9) mA cm⁻² in CO reduction reactions (CORR). When run at 300 mA cm⁻², the tested CORR system maintained stable n-propanol electrosynthesis for 100 hours.

The electrocatalyst includes Cu that can be deposited onto the substrate using various techniques, such as spray coating or sputtering, to form a coated substrate. The substrate can be a porous gas diffusion membrane, which can be composed of various materials, such as polytetrafluoroethylene (PTFE). The Cu layer can be in the form of Cu nanoparticles that are spheroid in morphology. The Cu layer can have a thickness between 30 microns and 100 microns provided on the substrate. The substrate can be composed of various materials that are hydrophobic, such as polymeric materials, carbon, or combinations thereof.

Once the Cu layer is formed, the coated substrate can be subjected to a first doping stage to dope the Cu with a first-stage dopant metal selected from Ruthenium (Ru), Rhodium (Rh), Iridium (Ir), Palladium (Pd), Gold (Au) and Platinum (Pt). The first doping stage can involve immersing the coated substrate into a doping solution that includes cations of the first-stage dopant metal and enables the doping by galvanic replacement of Cu atoms with the dopant atoms. The doping solution can be an aqueous solution of a chloride or nitrate of the first-stage dopant metal, e.g., RuCl₃, Ru(NO₃)₃. This doping compound can be provided in the solution at a concentration between 1 micromole/L to 10 millimole/L and the solution temperature can be 25 degrees Celsius to 80 degree Celsius. The immersion time, temperature and concentration of the doping solution are factors that can impact the degree of doping. The solution can be stirred or not during the galvanic replacement process. Once a target doping is achieved for the first-stage dopant, the substrate is removed from the doping solution. The partially doped material can be prepared for the second doping stage by washing (e.g., with deionized water) and drying which can be done using N₂ gas or another inert gas, for example.

The first doping stage is followed by a second doping stage to incorporate Ag into the electrocatalyst. Thus, the substrate with a partially doped Cu coating is immersed in a second doping solution that includes Ag cations to enable galvanic replacement of Cu atoms with Ag atoms. The doping solution can be an aqueous solution of a nitrate of the second-stage dopant metal, e.g., AgNO₃. This doping compound can be provided in the solution at a concentration between 1 micromole/L to 10 millimole/L and the solution temperature can be 25 degrees Celsius to 80 degree Celsius. The immersion time, temperature and concentration of the Ag doping solution are factors that can impact the degree of Ag doping. Once a target doping is achieved for Ag, the substrate is removed from the doping solution. The co-doped material can be further treated after the second or final doping stage by washing (e.g., with deionized water) and drying which can be done using N₂ gas.

In an alternative implementation, instead of first depositing the Cu on the substrate, the Cu metal is initially doped to form the co-doped multi-metallic material which is then deposited onto the substrate. For example, one can use the aqueous solution of nitrate or chloride of Cu, first dopant (e.g., Ru, Rh, Ir, Pt, Au, and Pd), second dopant (e.g., Ag), and add reducing agents (e.g., ascorbic acid, sodium borohydride) to obtain tri-metallic catalysts by one-step wet synthesis.

The two-stage method for making the co-doped electrocatalyst leverages the difference in reduction potential of the two dopant metals and Cu for enhancing the manufacturing process. For example, if the Ag were to be doped first, then the Ru could replace not only Cu atoms but also Ag atoms in the subsequent doping step. However, by doping Cu with Ru first, the difference in reduction potential between Ag and Ru facilitates doping of Ag without notable replacement of Ru atoms. Thus, the two-stage method facilitates controlled co-doping and efficient use of materials for making the co-doped electrocatalyst. It is noted in this regard that a two-stage doping method could be applied to other metals to provide multi-doped metallic materials by leveraging the differences in reduction potential to provide ordered doping steps.

It is also noted that the electrocatalyst could also be doped with three or more metals, and the manufacturing process can be adapted accordingly. For example, where the Cu is doped with Ag as well as two or more secondary dopant metals selected from Ru, Rh, Pt, Pd, Au and Ir, the order or staged doping can be based on the reduction potential of the dopant metals, as explained above. As mentioned above, soluble salts of the metals, such as chloride and/or nitrate salts, can be used to make the doping solutions.

In another alternative implementation, the method can be performed where the Cu is doped with Ag and secondary dopant metal simultaneously or in the reverse order as described above, although such methods may not be as efficient as the two-stage method described above. For simultaneous doping, the Ag—Ru doping solution could be prepared to provide metal cation concentrations tailored to provide the target doping levels for the respective dopant metals in the Cu.

In a further alternative method, the multi-metallic electrocatalyst can be produced by a technique other than galvanic replacement. For example, the material could be produced via one-step synthesis where Cu, Ag as well as Ru, Rh, Ir, Pd, Au and/or Pt cations in a solution are reduced and then removed and deposited onto a substrate.

In terms of target doping levels, in some implementations the co-doped multi-metallic material is provided with a first-stage dopant concentration between 0.5 wt % and 10 wt %, or 1 wt % and 3 wt %, which could be measured with XPS. Thus, the concentration of Ru, Rh, Pd, Pt, Au, or Ir would be 0.5 wt % to 10 wt % of the overall mass of the co-doped multi-metallic material. It is also possible to include more than one of these secondary dopant metals, in which case the total concentration of these dopants could be within the range of 1 wt % and 20 wt %. The first-stage dopant concentration can also be between 0.5 wt % and 10 wt %.

In some implementations, the co-doped multi-metallic material has a Ag concentration between 1 wt % and 10 wt %, between 2 wt % and 9 wt %, between 3 wt % and 5 wt %, or about 4 wt %, which could be measured with XPS. Alternative Ag concentrations can also be provided.

Furthermore, in some implementations, the co-doped multi-metallic material can have a first-stage dopant to Ag ratio between 1:1 and 1:10, between 1:2 and 1:7, between 1:3 and 1:5, or above 1:4, which could be measured with XPS. It is also noted that the concentration of the dopant metals can be provided based on functionality, such as the impact on CO adsorption, C1-C1 coupling, C1-C2 coupling, certain reaction kinetics, stability, and selectivity at certain operating conditions (e.g., high current densities) of the electrochemical cell. Thus, the concentrations can be adjusted outside or within the above-mentioned ranges to achieve one or more of these functionalities. In addition, the concentration of the secondary dopant can be provided to achieve increased performance compared to both Cu-only catalysts and Ag-only-doped Cu catalysts at certain electrocatalytic operating conditions, e.g., high current densities.

In some implementations, the nanoparticles of the co-doped multi-metallic material have an average size between about 20 nm and about 200 nm, or between about 70 nm and about 150 nm, measured based on SEM or TEM imaging. The co-doped material can have substantially the same morphology and size of nanoparticles as the Cu material pre-doping, and can therefore depend on the method of depositing or providing the Cu material.

The present work screened several catalysts through experimentation and found that the tri-metallic electrocatalyst Ag—Ru—Cu provided the best performance, and also that Ag—Rh—Cu tri-metallic electrocatalyst provided enhanced performance compared to Cu and bimetallic catalyst Ag—Cu. For example, compared to Ag—Cu bimetallic catalyst, higher selectivity toward total C2+ products and n-propanol was found for Ag—Ru—Cu tri-metallic electrocatalysts at high current density (above 100 mA cm⁻²). Compared to previous Ag—Cu bimetallic catalyst work, various performance variables were improved using the Ag—Ru—Cu tri-metallic electrocatalyst, including selectivity, production rate, and stability. Apart from the catalyst material itself, a membrane electrode assembly (MEA) was also used and it was found to improve the stability (above 100 hours for n-propanol production) compared to flow cell tests used for Ag—Cu work.

### Experimentation & Additional Information

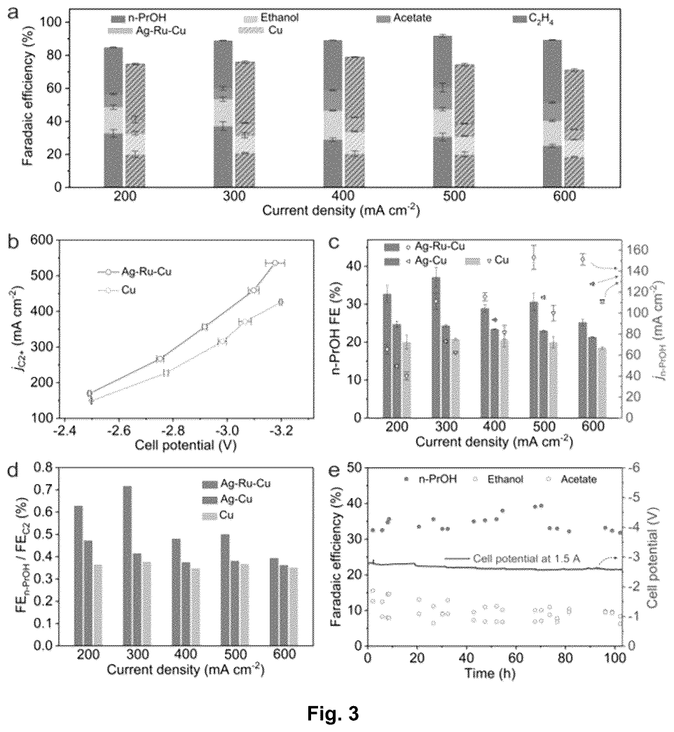

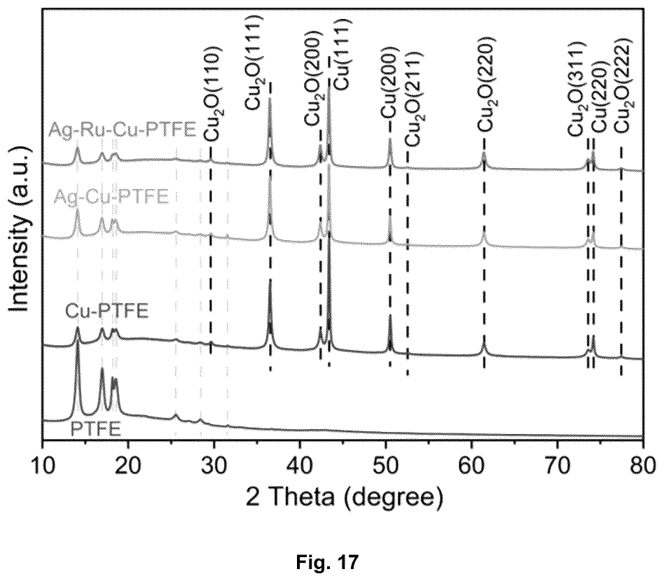

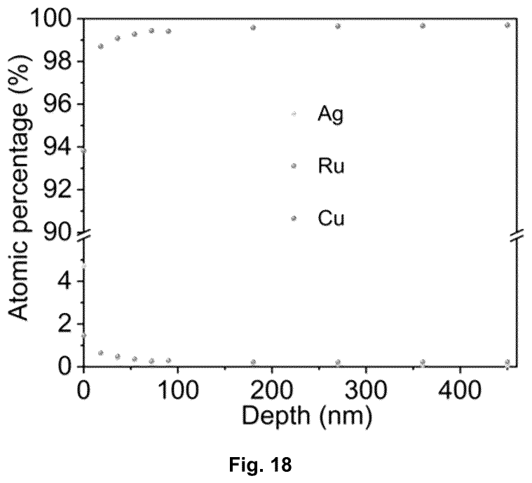

The present work sought to realize Ag—Ru—Cu catalysts experimentally. We first spray-coated a layer of commercial Cu nanoparticles onto a gas diffusion layer consisting of a porous polytetrafluoroethylene (PTFE) nanofiber substrate with sputtered 50 nm Cu layer on the surface (FIG. 16a). Then, the work prepared the Ag—Ru—Cu catalyst (FIG. 2, a and b, and FIG. 16b) via a two-step galvanic replacement between Cu and RuCl₃ and then between Cu and AgNO₃ driven by the difference in the reduction potentials of Ru vs. Cu and Ag vs. Cu (34-36), respectively. There was no observable morphology difference between the prepared Ag—Ru—Cu and the pristine Cu based on scanning electron microscopy (SEM) (FIG. 16). Energy-dispersive X-ray spectroscopy (EDS) elemental mapping reveals that Ag, Ru, and Cu elements are evenly distributed in the Ag—Ru—Cu nanoparticles (FIG. 2c). In the powder X-ray diffraction (XRD) patterns of the Cu and Ag—Ru—Cu electrodes, the work observed peaks of Cu₂O ascribed to the partial oxidation of Cu nanoparticles in the air during the electrode preparation (FIG. 17). High-resolution X-ray photoelectron spectroscopy (XPS) further confirms the existence of Cu, Ag, and Ru in the nanoparticles (FIG. 2, d to f). The atomic percentages of Ag and Ru in the electrode surface are approximately 4% and 1%, respectively, as determined using XPS.

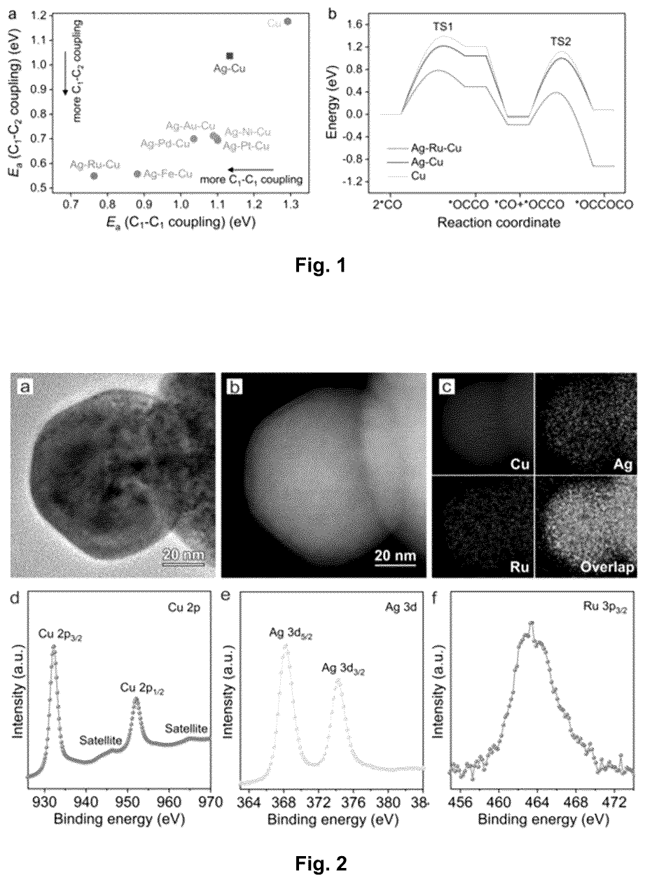

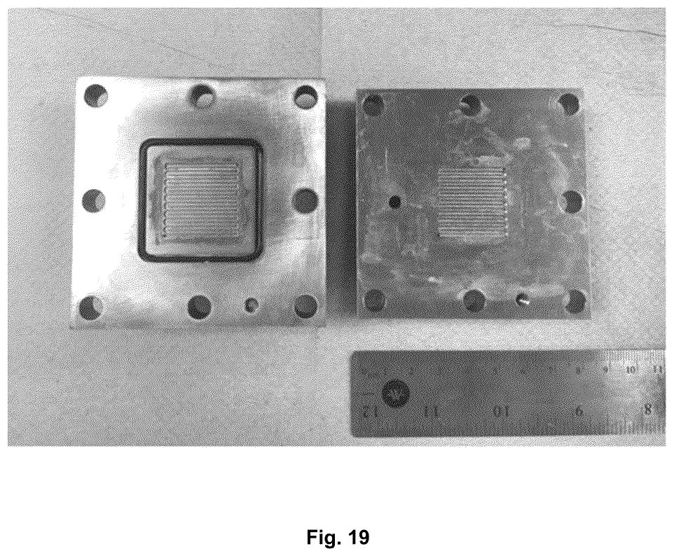

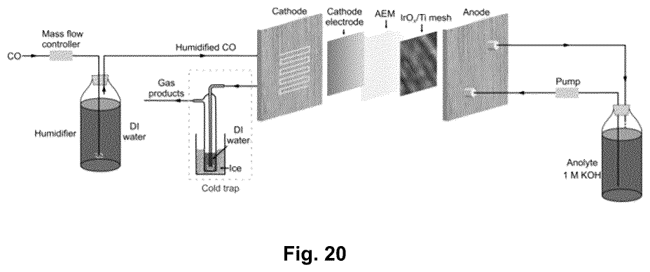

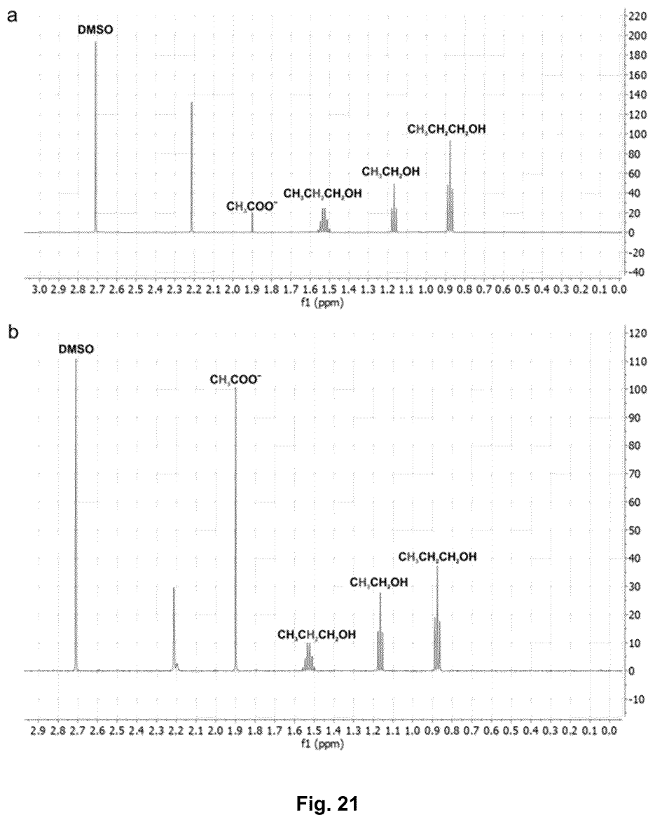

The CORR performance was evaluated in a membrane electrode assembly (MEA) reactor with both cathode and anode electrodes of 5 cm² geometric area (FIGS. 19 and 20), a scalable reactor enabling operation at conditions relevant to industrial electrolyzers (37). FIG. 3a displays the FEs of C₂₊ products (ethylene, ethanol, acetate, and n-propanol) on Ag—Ru—Cu and Cu electrodes during CORR in the current density range of 200-600 mA cm⁻². Both FE values of total C₂₊ products and n-propanol on Ag—Ru—Cu electrodes are higher than those on Cu electrodes (table S5 and FIG. 21), consistent with our DFT prediction (FIG. 1). In the regime of 300-600 mA cm⁻², the total FEs for C₂₊ products are up to ˜90% and the highest partial C₂₊ current density reaches 536 mA cm⁻² for the Ag—Ru—Cu electrodes (FIG. 3c). Specially, under a current density of 300 mA cm⁻², we achieve a record n-propanol FE of (37±3) % on Ag—Ru—Cu electrodes—1.8× higher than that on Cu electrodes—at a production rate of (111±9) mA cm⁻² associated with a full cell potential of (−2.75±0.01) V without ohmic loss correction, representing a full-cell EE of 14% for n-propanol.

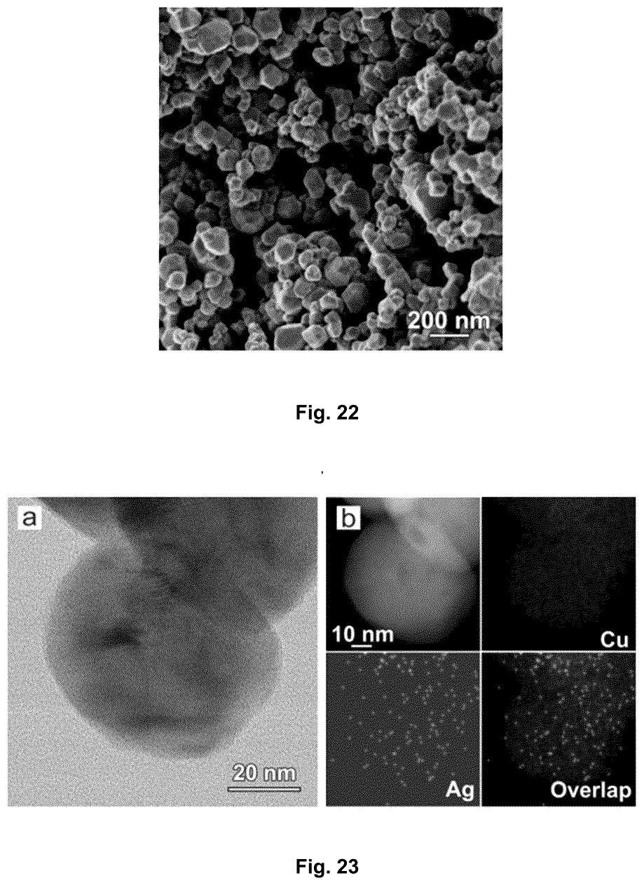

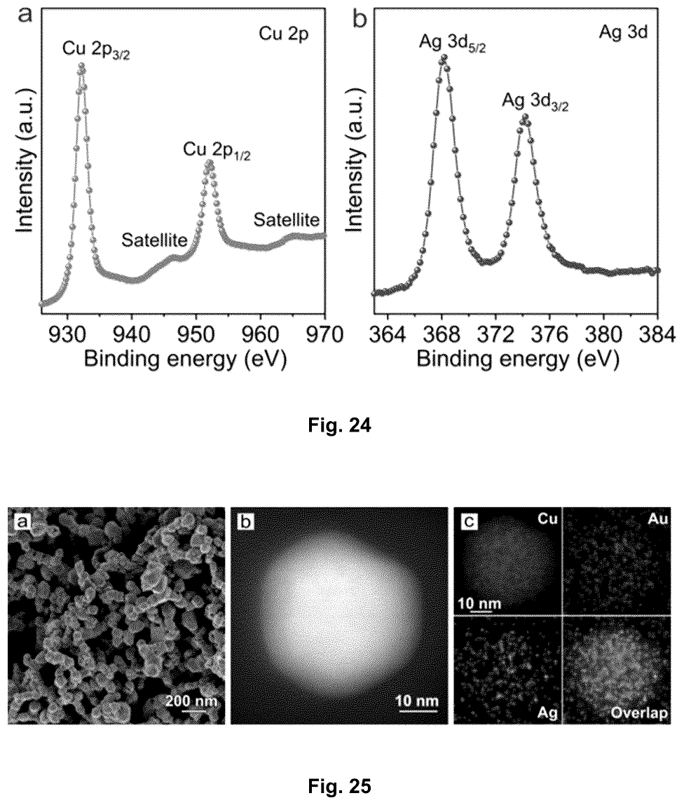

To explore further the effect of co-doping of Ag and Ru in Cu on the CORR performance, we also prepared Ag-doped Cu (denoted Ag—Cu) electrodes and measured their CORR performance for comparison. The Ag—Cu electrodes were prepared via a similar galvanic replacement approach and the atomic percentage of Ag doping in Cu on the electrode surface was also approximately 4%, as determined by XPS (FIGS. 22 to 24). Under the same current densities, the total FEs of C₂₊ products on the Ag—Cu electrodes are higher than those on Cu electrodes, but lower than those on Ag—Ru—Cu electrodes (table S5), indicating that the Ag doping in Cu favors C₁-C₁ coupling for C₂₊ products relative to Cu and the co-doping of Ag and Ru further enhances the C₂₊ selectivity. The n-propanol FEs on different electrodes follow the sequence Ag—Ru—Cu>Ag—Cu>Cu (FIG. 3c), suggesting that co-doping of Ag and Ru in Cu also promotes the step of C₁-C₂ coupling vs. Ag—Cu and Cu, in agreement with our calculations (FIG. 1). At 500 mA cm⁻², the highest partial n-propanol current density on the Ag—Ru—Cu electrodes is (153±12) mA cm⁻², representing 1.3× and 1.5× improvement relative to the Ag—Cu and Cu electrodes, respectively (FIG. 3c).

To evaluate the selectivity toward n-propanol vs. C₂ products in CORR, we compare the ratios of n-propanol FE to total C₂ FE (FE_{n-PrOH}/FE_{C2}) on different electrodes. Relative to the Ag—Cu and Cu electrodes, the Ag—Ru—Cu electrodes display higher FE_{n-PrOH}/FE_{C2} (FIG. 3d), further suggesting that the co-doping of Ag and Ru in Cu promotes the coupling reaction between C₁ and the C₂ intermediates.

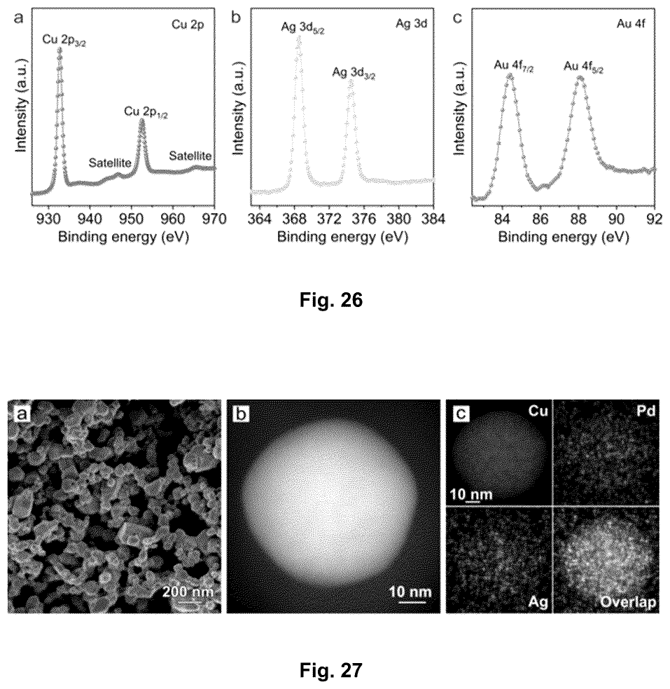

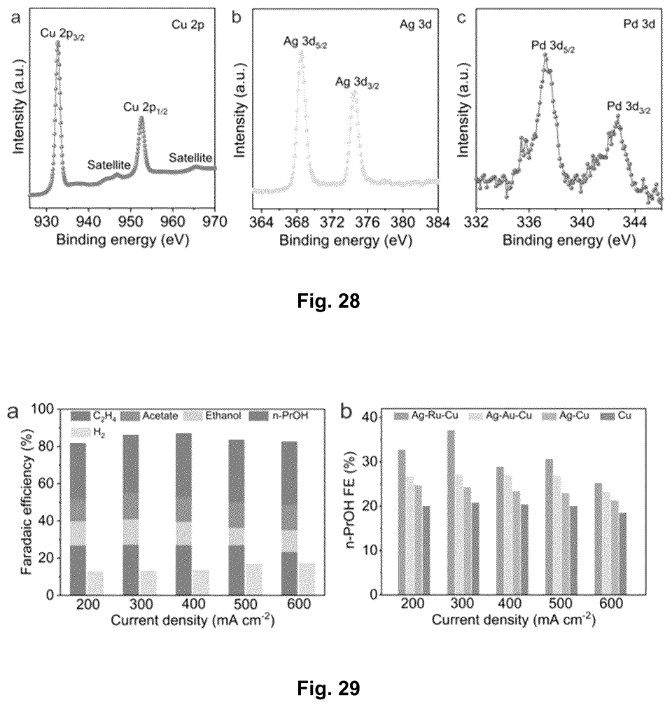

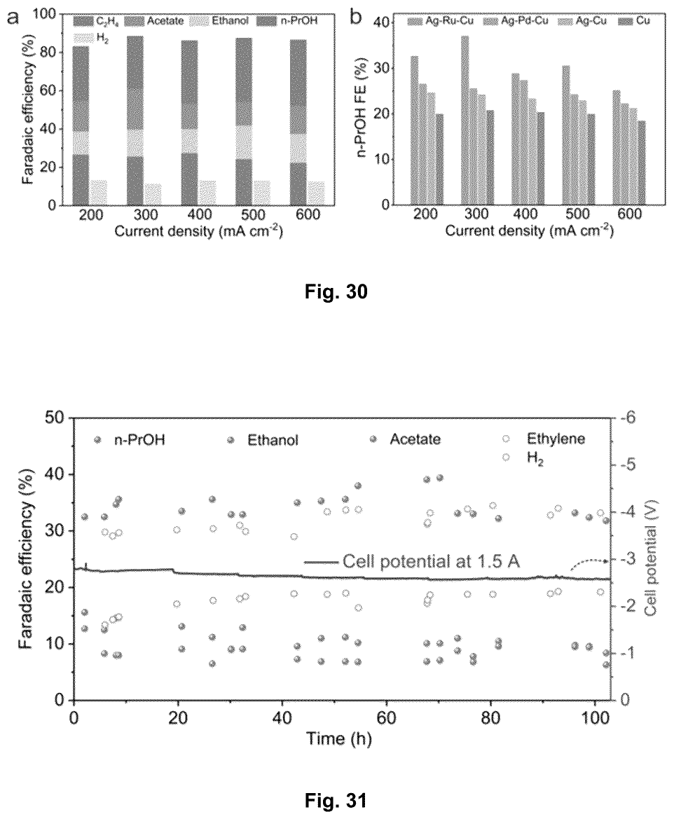

As controls, the work also prepared Ag—Au—Cu and Ag—Pd—Cu electrodes (FIGS. 25 to 28) and measured their CORR performance (FIGS. 29 and 30). By comparing the n-propanol FEs under the same current densities among different electrodes, the work found that Ag—Au—Cu and Ag—Pd—Cu electrodes exhibit lower n-propanol selectivity than Ag—Ru—Cu electrodes, but higher n-propanol selectivity relative to Ag—Cu and Cu electrodes, in agreement with calculations.

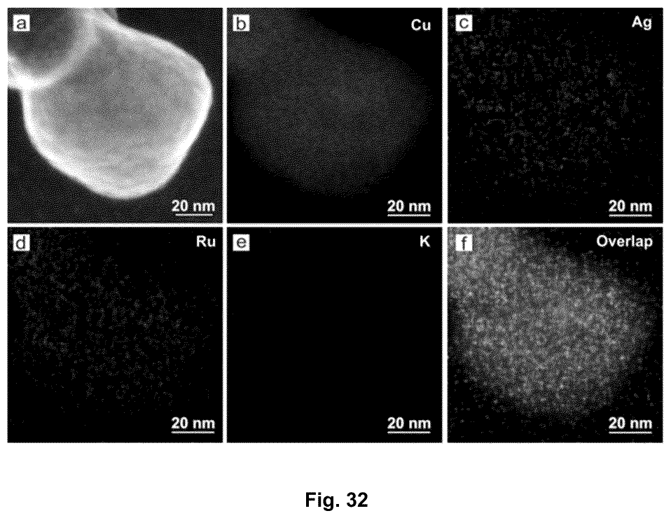

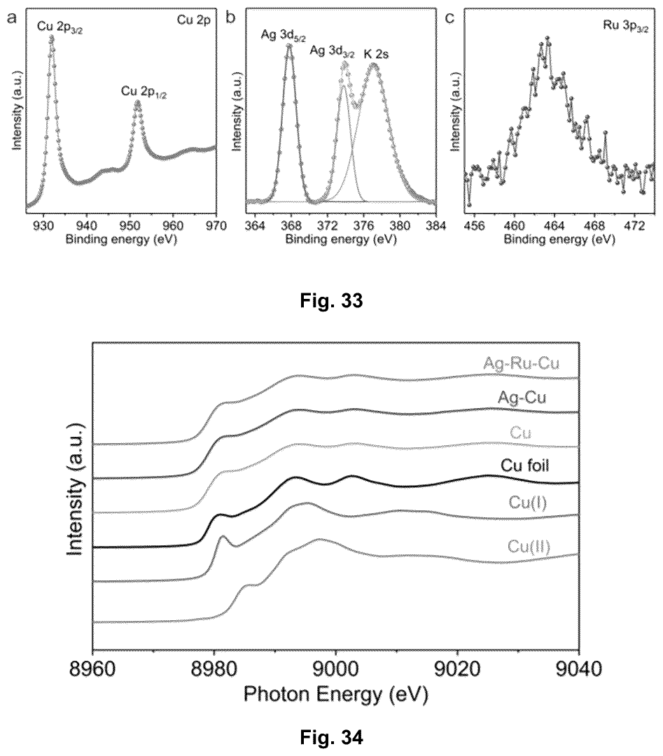

By selecting the electrolysis condition with the highest n-propanol selectivity, the work further evaluated the CORR stability on the Ag—Ru—Cu electrode at 1.5 A (300 mA cm⁻²) in the MEA reactor (FIG. 3e and FIG. 31). The system maintained a stable full-cell potential of (−2.64±0.07) V during the CORR measurement. Throughout the 102 hours of CORR operation, a n-propanol FE above 32% was maintained on the Ag—Ru—Cu electrode. The TEM, EDX, and XPS analyses on the post-reaction catalyst reveal that the Ag—Ru—Cu catalyst retains its structure following operational stability measurement (FIGS. 32 and 33).

The work carried out in situ X-ray absorption spectroscopy (XAS) studies at the Cu K-edge to investigate the chemical state of Cu in different catalysts during CORR. The Cu K-edge X-ray absorption near-edge structure (XANES) spectra exhibit that, under the current density of 300 mA cm⁻², the average valance states of Cu in Ag—Ru—Cu, Ag—Cu, and Cu are all zero during CORR (FIG. 34), demonstrating that the product selectivity on Ag—Ru—Cu, Ag—Cu, and Cu catalysts in CORR are related to metallic Cu; and excluding the possibility that their different CORR performance might arise from the difference in Cu valance states.

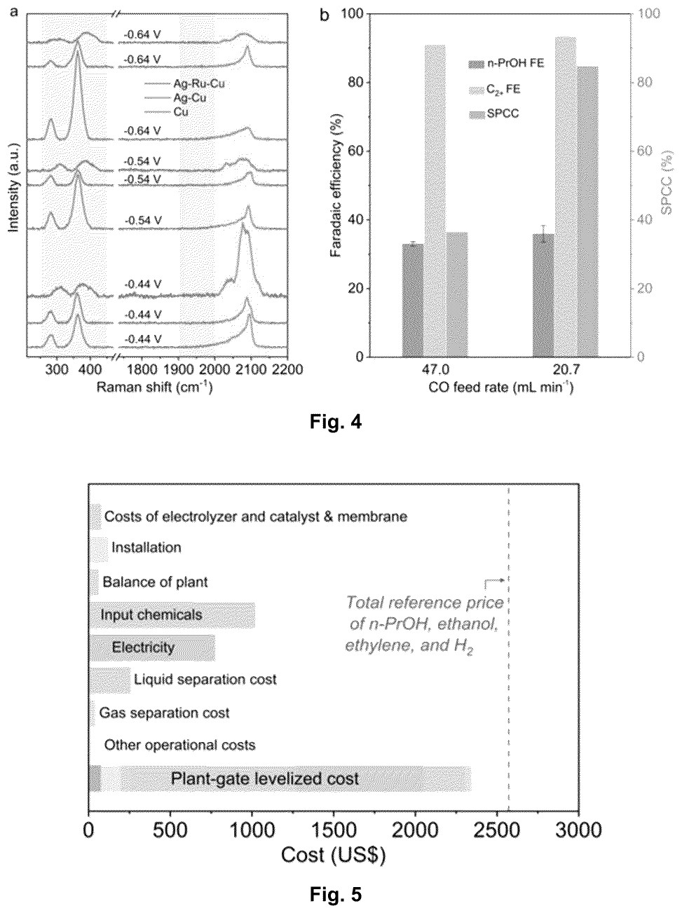

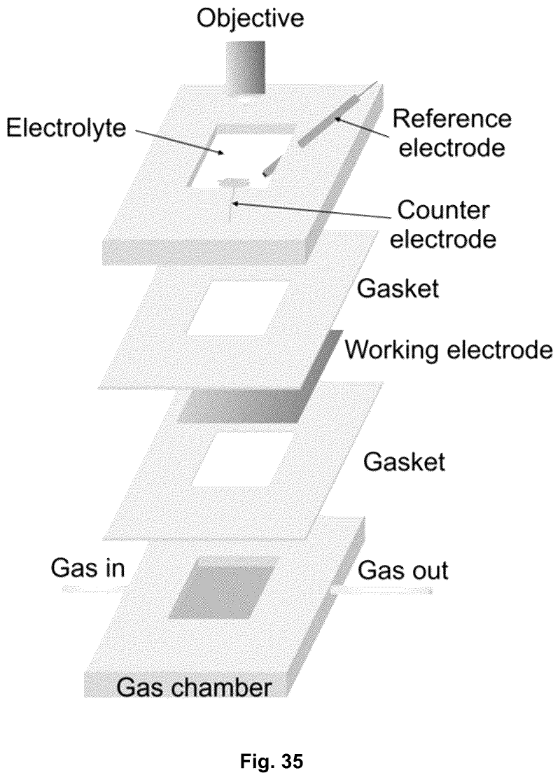

To gain insight into C—C coupling among the different catalysts, the work also performed in situ Raman spectroscopy measurements during CORR under different potentials (FIG. 4a and FIG. 35). The bands in the range of 1900-2150 cm⁻¹ arise from the C≡O stretching of the adsorbed CO on metal sites, wherein the regions below and above 2000 cm⁻¹ are attributed to the bridge-bound CO (CO_{bridge})—which is not an on-pathway intermediate in CORR—and the atop-bound CO (COₐₜₒₚ), respectively. Relative to Cu, the C≡O stretching bands on Ag—Ru—Cu and Ag—Cu are only from COₐₜₒₚ on the surface, indicating that the adsorbed CO on Ag—Ru—Cu and Ag—Cu is in a more favorable configuration for further reaction to C₂₊ products compared to Cu.

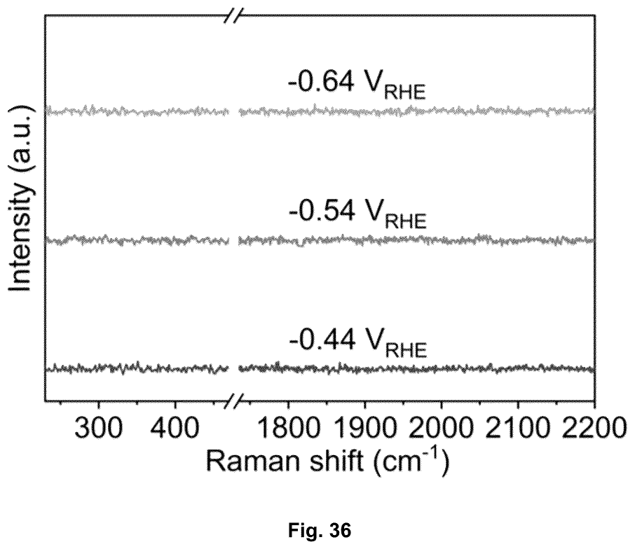

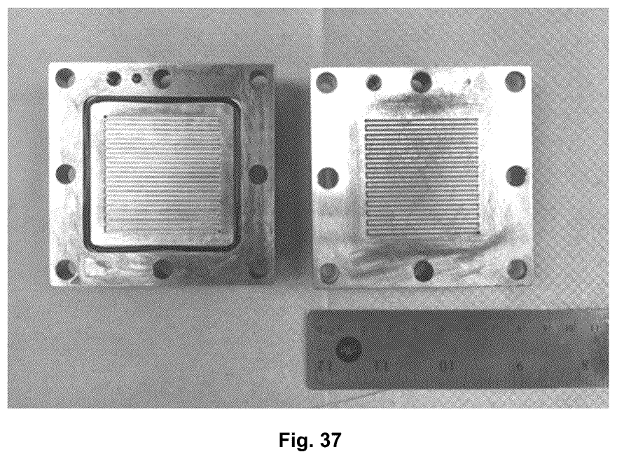

In the Raman spectra, the bands located at ˜283 and ˜363 cm⁻¹ are associated with frustrated rotation of *CO on Cu and Cu—CO stretching, respectively. Under the same applied potentials, one observes a blueshift of the Cu—CO stretching band on Ag—Ru—Cu relative to Ag—Cu and Cu (FIG. 4a). The blueshift of the Cu—CO stretching band indicates that a stronger Cu—CO bonding on the Ag—Ru—Cu surface compared to the Ag—Cu and Cu surfaces, which might favor the step of C—C coupling and the subsequent generation of C₂₊ products. As controls, the work also acquired in situ Raman spectra with Ar-saturated KOH electrolyte to confirm that the peaks in the regions marked orange, blue, and yellow arise from the conditions of CORR (FIG. 4a and FIG. 36).

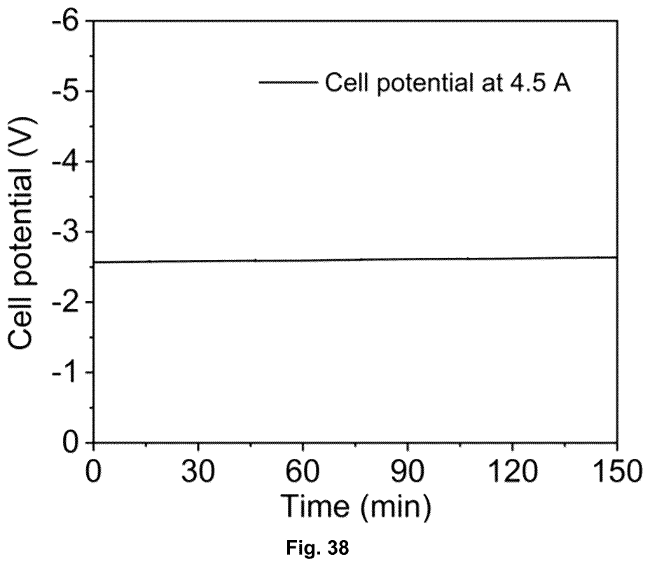

The work investigated the extent to which n-propanol electrosynthesis can be scaled, seeking to increase the active area to 15 cm² (A=15 cm²) (FIG. 4b, FIGS. 37 and 38, and table S6). The work achieved, at a current density of 300 mA cm², n-propanol FE of (36±3) % and a C₂₊ FE of 93% at a full-cell potential of (−2.60±0.02) V (again without ohmic loss correction); this corresponds to a full-cell energy efficiency (EE) of 37% for all C₂₊ products. To reduce the energy penalty of unreacted CO and gas product separation after reaction, we pursued a high single-pass CO conversion (SPCC) in the system. We lowered the CO feed rate and observed that Ag—Ru—Cu catalysts could retain similar product selectivities at low CO feed rate; as a result, we achieved a SPCC as high as 85% for C₂₊ products.

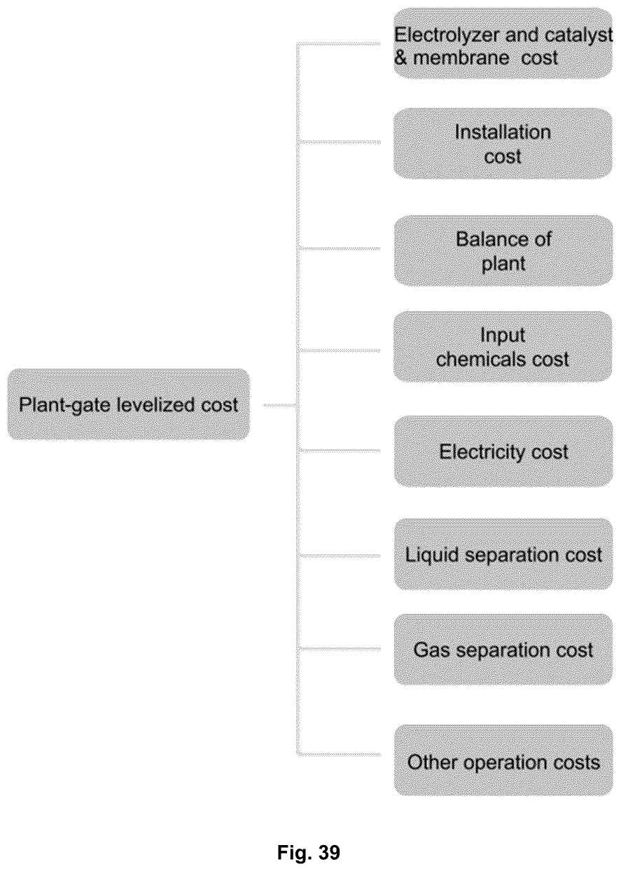

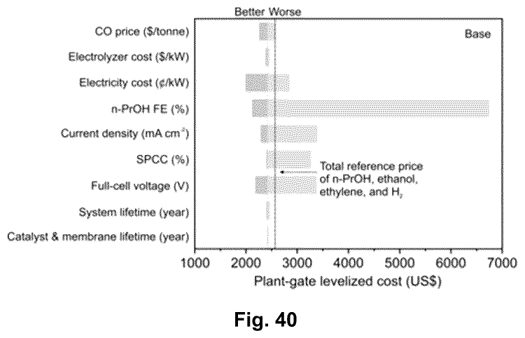

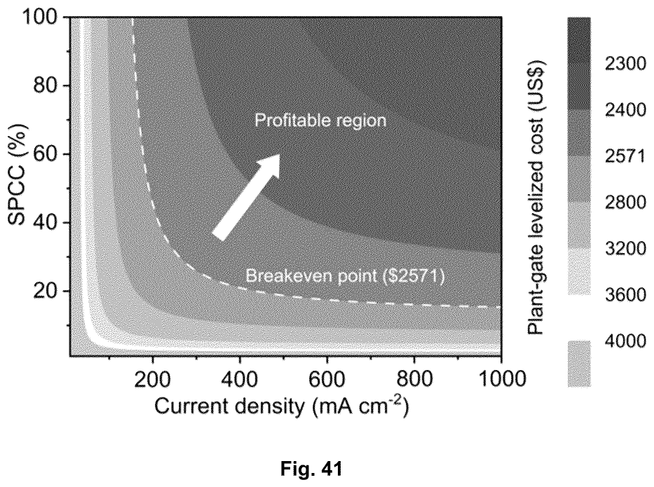

As the Ag—Ru—Cu electrodes deliver the highest n-propanol selectivity at 300 mA cm⁻², we performed a TEA based on their CORR performance (FIG. 5, FIGS. 39 and 40, and table S7), where n-propanol, ethanol, ethylene, and H₂ products are considered as the products for sale in the calculation. The work accounted for the cost of separation, including the separation of liquid products from one another, and gas products from one another; the costs for the electrolyzer, catalyst, membrane, installation, balance-of-plant, input chemicals, and electricity; and the other operational costs (such as labor and maintenance). Sensitivity analysis reveals that the plant-gate levelized cost depends most importantly on electricity cost and on electrochemical performance parameters such as n-propanol FE, current density, SPCC, and full-cell potential (FIG. 40 and table S7). Further calculation reveals that, with an n-propanol FE of 36%, the renewable-electricity-powered n-propanol electrosynthesis become profitable only when the current density is higher than 150 mA cm⁻² and SPCC is above 15% (FIG. 41).

The TEA calculation—based on the CORR performance data at 300 mA cm⁻² in the A=15 cm² MEA electrolyzer—shows that the plant-gate levelized cost for one tonne of n-propanol, plus the corresponding quantity of ethanol, ethylene, and H₂ produced at 300 mA cm⁻² on Ag—Ru—Cu electrodes, is projected to be less than the sum of their reference prices (FIG. 5). This result suggests that the CORR on the Ag—Ru—Cu electrodes under the above conditions shows promise.

A computational study of candidates was also performed and included Ag—Ru-co-doped Cu (Ag—Ru—Cu) catalysts. These catalysts enable a n-propanol FE of (37±3) % at a partial current density of (111±9) mA cm⁻², and a full-cell energy efficiency (EE) of 14% during CORR. We report stable n-propanol electrosynthesis over 100 hours at 300 mA cm⁻².

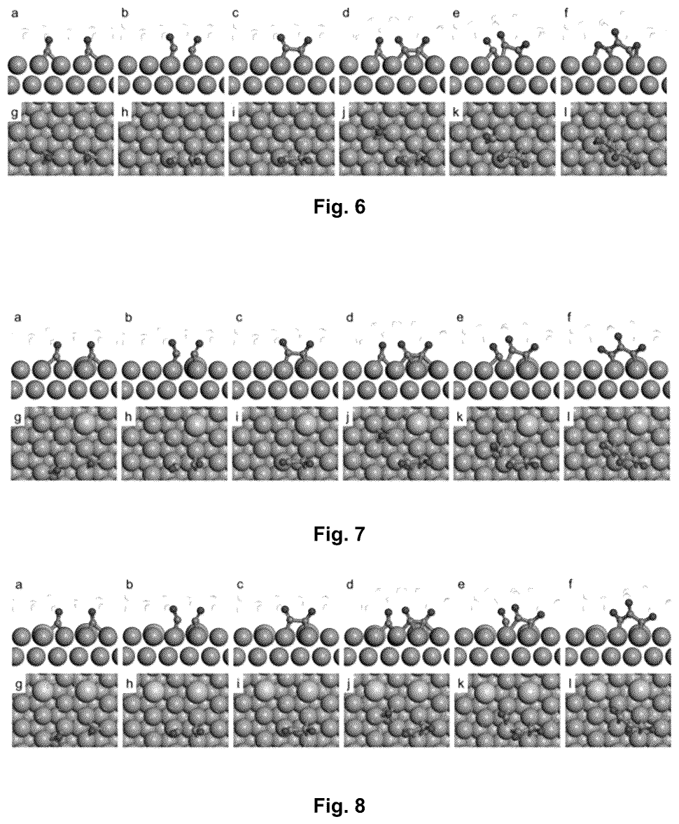

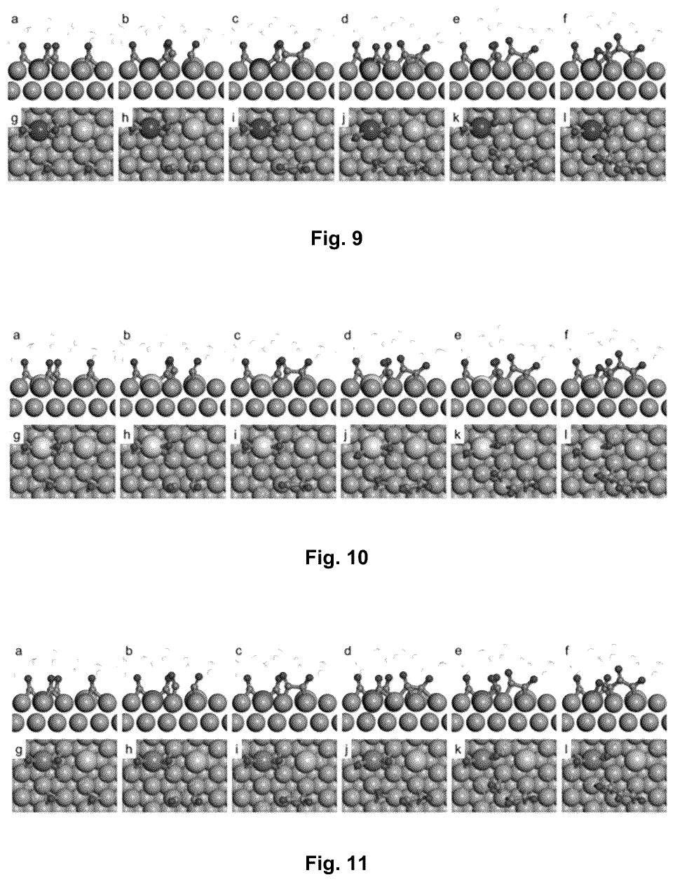

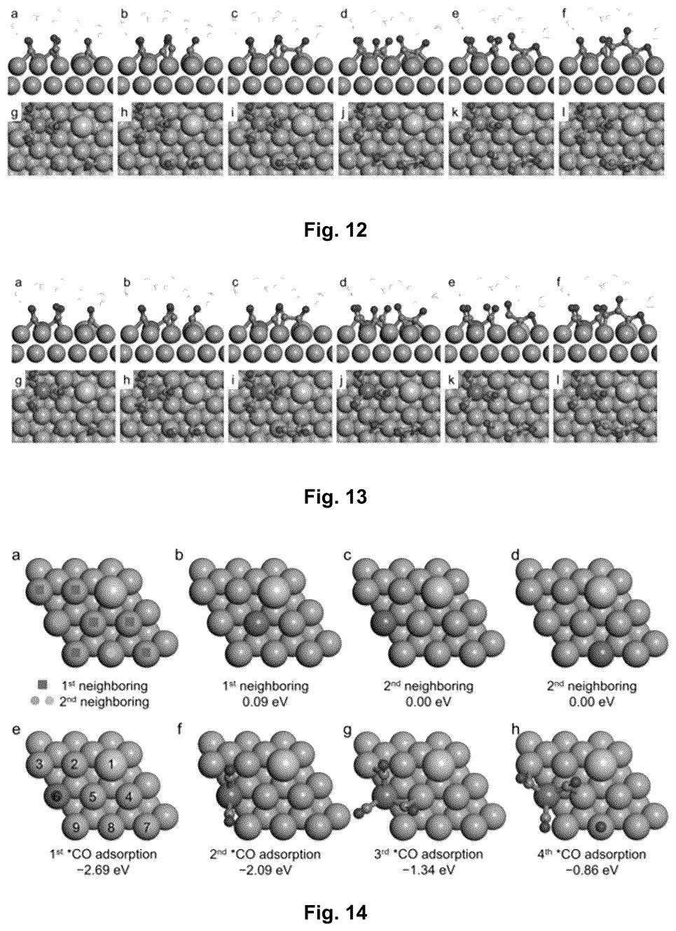

This work also used density functional theory (DFT) to screen catalyst systems considering their propensity to catalyze the C₁-C₁ and C₁-C₂ coupling steps. Ag-doped Cu (denoted Ag—Cu) is an experimentally reported bimetallic catalyst that favors selectivity to n-propanol compared to Cu. We therefore considered several Ag—X-co-doped Cu (denoted Ag—X—Cu, where X represents an additional metal) catalyst systems for the theoretical screening (FIG. 1a, FIGS. 6 to 14, and tables S2 and S3). Based on prior studies, we calculated the reaction energies of the *CO dimerization (*CO+*CO→*OCCO) and the coupling between *CO and *OCCO intermediates (*CO+*OCCO→*OCCOCO) on different Ag—X—Cu catalyst systems, then applied them as predictors for the activities of C₁-C₁ and C₁-C₂ couplings, respectively. Of the catalyst systems screened, Ag—Ru—Cu requires the lowest reaction energies for C₁-C₁ and C₁-C₂ couplings (FIG. 1a), suggesting that Ag—Ru—Cu to be the most promising candidate favoring C₃ formation from CO.

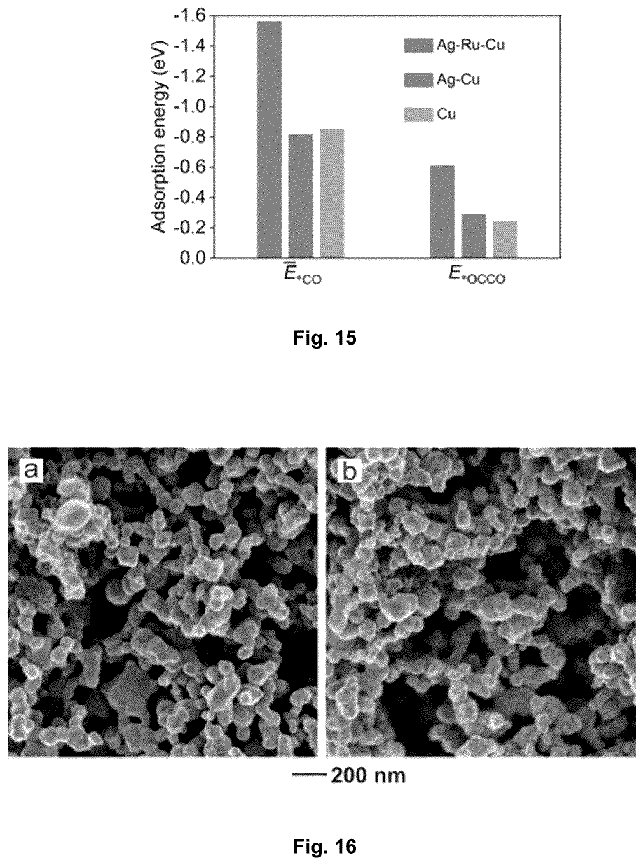

We further compared the adsorption energies of *CO and *OCCO, the possible key reaction intermediates for the C₁-C₁ and C₁-C₂ coupling steps, on Ag—Ru—Cu with those on Ag—Cu and Cu (FIG. 1b and FIG. 15). Relative to Ag—Cu and Cu, the higher average *CO adsorption energy on Ag—Ru—Cu demonstrates that CO molecules are more readily adsorbed on Ag—Ru—Cu. Specifically, the co-doping of Ag and Ru in Cu induces CO adsorption near the C₁-C₁ and C₁-C₂ coupling sites and, thus, results in higher *CO coverage on the surface compared to Ag—Cu and Cu, which may promote multiple C—C coupling steps (tables S2 and S4). Additionally, the adsorption energy of the key C₂ intermediate for C₁-C₂ coupling on Ag—Ru—Cu is higher than that on Ag—Cu and Cu; this may reduce the desorption of C₂ intermediates from the Ag—Ru—Cu surface and the subsequent formation of C₂ products, thus increasing the residence of C₂ intermediates necessary for C₃ generation. These calculations, taken together, suggest Ag—Ru—Cu has the potential to improve C₃ selectivity at high production rates.

Additional information is provided below with respect to the field and applications of embodiments of the technology described herein. Electrolysis powered using renewable electricity provides an attractive route to upgrade C1 feedstocks, such as carbon dioxide and carbon monoxide, to valuable fuels and chemicals. N-propanol, a higher alcohol with high energy density, is a desirable product in carbon monoxide electroreduction (CORR). Using existing techniques, the performance of this reaction-including selectivity, production rate, and stability-remains low relative to requirements for practical applications. Catalysts that simultaneously promote multiple carbon-carbon (C—C) coupling steps, stabilize C2 intermediates, and promote CO adsorption, can facilitate enhanced n-propanol production at a high reaction rated.

The electrochemical reduction of carbon dioxide (CO₂RR) to valuable fuels and chemical feedstocks offers a promising route to store intermittent renewable electricity. Progress has been achieved in the electrosynthesis of C₁ and C₂ products with high selectivity at commercially relevant current density (>100 mA cm⁻²) from CO₂RR. To date, CO₂RR studies have been performed predominantly under alkaline/neutral electrolytes with a local pH>7 at the catalyst surface during the reaction, leading to the partial consumption of CO₂ through bicarbonate/carbonate formation and added cost for CO₂ regeneration. The electrochemical reduction of carbon monoxide (CORR) following the CO₂ reduction to CO, a two-step cascade process, provides a solution to address the problem in direct CO₂RR. Owing to advances in solid oxide electrolysis cell technology, CO feedstock can now be produced from CO₂ at low cost with energy efficiency exceeding 90% at ˜200 mA cm⁻², furthering applications of CORR.

Among reported C₁-C₃ products in CORR, the C₃ alcohol n-propanol is particularly desirable in light of its high energy density and high octane number. It is suitable for use as engine fuel, as a solvent, and as the raw material for n-propyl acetate. Today n-propanol is manufactured mainly through the catalytic hydrogenation of propionaldehyde following the high-pressure production of propionaldehyde via the hydroformylation of ethylene with CO and H₂ under the condition of heat. This complex manufacturing process drives up cost and thus limits the overall size of the n-propanol market; yet, in light of its higher energy density, n-propanol could take the place of ethanol as a transportation fuel additive for which the market would grow if n-propanol could be efficiently produced. Hence, it is attractive to explore whether n-propanol could be generated efficiently through electrolysis using renewable electricity.

Technoeconomic analysis (TEA) has shown that reaction rates must at least exceed 100 mA cm⁻² for profitable CORR systems. Experimentally, prior CORR/CO₂RR systems with current densities above 100 mA cm⁻² have shown limited selectivity toward n-propanol, with a maximum Faradaic efficiency (FE) of 18% (table S1).

The generation of C₃ in CORR relies on C₁-C₁ coupling and the subsequent C1-C2 coupling. The key step branching the pathways to C₃ and C₂ products is identified as C—C coupling between C₁ and C₂ intermediates. To ensure the production of C₃ at high production rates, adequate C₂ intermediates must be formed and stabilized on the catalyst surface and, thus, be available to be coupled with adsorbed CO.

It was viewed that to promote C₃ selectivity at high production rates, a good catalyst will facilitate both the C₁-C₁ and the C1-C₂ coupling steps, stabilize C2 intermediates, and promote CO adsorption.

It is also noted that experiments were conducted with Ag—Rh—Cu tri-metallic electrocatalysts and enhanced performance was also observed with this implementation, although the tested Ag—Ru—Cu catalysts showed greater enhancement than the tested Ag—Rh—Cu catalysts.

The following section provides supplementary information regarding the present work including experimentation and examples:

### Materials and Methods

### Chemicals

Silver nitrate (AgNO₃, 99.0%), ruthenium (Ill) chloride hydrate (RuCl₃·xH₂O), and iridium (Ill) chloride hydrate (IrCl₃·xH₂O, 99.9%) were purchased from Sigma-Aldrich. Potassium hydroxide (KOH) was received from Caledon Laboratory Chemical. Anion exchange membrane (Fumasep FAA-3-50) and titanium mesh were received from Fuel Cell Store. Sustainion anion-exchange membrane was purchased from Dioxide Materials. The anion exchange membranes were activated in 1 M KOH aqueous solution for 24 hours followed by washing with deionized (DI) water before use. Copper target (99.999%) was purchased from Kurt J. Lesker company. PTFE membrane with an average pore size of 450 nm was received from Beijing Zhongxingweiye Instrument Co., Ltd. All chemicals were used as received. The aqueous solutions were prepared using DI water with a resistivity of 18.2 MΩ cm.

### Preparation of Electrodes

We first prepared a gas diffusion electrode by sputtering 50 nm thickness Cu (Cu target, sputtering rate: ˜0.6 Ås⁻¹) on a piece of PTFE membrane using a magnetron sputtering system. Cu nanoparticles (Sigma-Aldrich) was dispersed in a mixture of methanol and Nafion perfluorinated resin solution (5 wt. % in mixture of lower aliphatic alcohols and water; Sigma-Aldrich) with a volume ratio of 100:1 under ultrasonication for 30 min to prepare a suspension with a Cu concentration of 9.9 mg mL⁻¹. The suspension was spray-coated on the gas diffusion electrode with a Cu nanoparticle loading of 6 mg cm⁻² to prepare the Cu electrode.

To prepare Ag—Ru—Cu electrode, we first immersed the prepared Cu electrode in a N₂-saturated 5 mmol L⁻¹ RuCl₃ aqueous solution at 65° C. for 20 min and then immersed the electrode in a N₂-saturated 5 mmol L⁻¹ AgNO₃ aqueous solution at 65° C. for 2 h. The Ag—Cu electrode was prepared using a similar galvanic replacement approach: the prepared Cu electrode was immersed in a N₂-saturated DI water at 65° C. for 20 min and then was immersed in a N₂-saturated 5 mmol L⁻¹ AgNO₃ aqueous solution at 65° C. for 2 hours. For Ag—Au—Cu electrode, the prepared Cu electrode was immersed in a N₂-saturated 5 μmol L⁻¹ HAuCl₄ aqueous solution at 40° C. for 15 min and then was immersed in a N₂-saturated 5 μmol L⁻¹ AgNO₃ aqueous solution at 65° C. for 2 h. For Ag—Pd—Cu electrode, the prepared Cu electrode was immersed in a N₂-saturated 5 μmol L⁻¹ H₂PdCl₄ aqueous solution at 65° C. for 20 min and then was immersed in a N₂-saturated 5 μmol L⁻¹ AgNO₃ aqueous solution at 65° C. for 2 h.

The iridium oxide supported on titanium mesh (IrOₓ/Ti mesh) was used as the anode catalyst which was prepared by a dip coating and thermal decomposition method reported.

### Materials Characterization

SEM images were taken using Hitachi FE-SEM SU5000 microscope. HAADF-STEM images and the corresponding EDX elemental mapping were taken using a Hitachi HF-3300 microscope at 300 kV. Structural characterization of cathodes was carried out using XRD (MiniFlex600) with Cu-Kα radiation. The surface compositions of electrodes were determined by XPS (model 5600, Perkin-Elmer) using a monochromatic aluminum X-ray source.

In situ Raman measurements were performed using a Renishaw inVia Raman Microscope in a modified flow cell and a water immersion objective (63×) with a 785 nm laser (FIG. 35). The different cathode catalysts prepared, Ag/AgCl reference electrode (3 M KCl, BASi), and platinum coil were used as the working electrodes, reference electrode, and counter electrode, respectively. 1 M KOH aqueous solution was used as the electrolyte. CO or Ar was continuously supplied to the gas chamber during the measurement. The potentials in Raman measurement were converted to values with reference to RHE using the equation: E_{RHE}=E_{Ag/AgCl}+0.210 V+0.0591×pH.

In situ XAS measurement were conducted at the BL-17C in NSRRC. We measured the in situ XAS spectra at 300 mA cm⁻² during CORR using a flow cell reactor, a configuration the same as that used in a previous report. In the flow cell reactor, Ag/AgCl reference electrode (3 M KCl, BASi), Ni foam (1.6 nm thickness, MTI Corporation), and anion exchange membrane (Fumasep FAB-PK-130, Fuel Cell Store) were used as the reference electrode, anode, and membrane, respectively. 1 M KOH aqueous solution was used as the electrolyte and CO (Linde, 99.99%) was continuously supplied to the gas chamber during CORR. XAS data were processed using Athena and Artemis software included in a standard IFEFFIT package.

### Electrochemical Measurements

Without specification, all the CORR performance was measured in the A=5 cm² MEA electrolyzer (SKU: 68732; Dioxide Materials) (FIGS. 19 and 20). The MEA electrolyzer installation procedure is the same as that used in our previous report. For the A=15 cm² MEA electrolyzer (FIG. 37), the cathode electrode (3.875 cm×3.875 cm) was positioned on the cathode side and thus the activated Sustainion membrane (6.5 cm×6.5 cm) and an IrOₓ/Ti mesh anode electrode (3.875 cm×3.875 cm) were put on the top of the cathode successively; all were assembled in the MEA electrolyzer.

The MEA electrolyzer-based CORR measurement procedure is similar to that used in a previous report. CO gas (Linde, 99.99%) at different feed rates flowed to the humidifier with DI water continuously and was then supplied to the cathode chamber. Anolyte (1 M KOH aqueous solution) was introduced into the anode chamber and was circulated using a pump (10 mL min). Using an electrochemical station (AUT50783) equipped with a current booster (10 A), we evaluated the performance of the cathode electrode in two-electrode system at different current densities. The long-term operation test was also performed in the MEA electrolyzer and the anion exchange membrane (Fumasep FAB-PK-130) was used as the membrane. The products from cathode side went through a simplified cold trap which was used for separating liquid products and gas products. The gas products were tested by gas chromatograph (PerkinElmer Clarus 600). The liquid products were analyzed using NMR spectrometer (Agilent DD2 600 MHz) with dimethylsulfoxide (DMSO) as an internal standard. Liquid product FE was calculated by considering the total amount of the products collected from anode and cathode sides in the same period.

For different products A (A=n-propanol, ethanol, ethylene, and acetate), the full-cell EE for product A is calculated as follows:

$${EE}_{full - cell , A} = \frac{( 1.23 + ( - {E}_{A}^{o} ) ) \times {FE}_{A}}{- {E}_{full - cell}} ,$$

where E_{A}^{o} is the thermodynamic potential of CO to product A (E_{n-PrOH}^{o}=0.20 V vs. RHE; Eₑₜₕₐₙₒₗ^{o}=0.178 V vs. RHE; E_{ethylene}^{o}=0.17 V vs. RHE; E_{acetate}^{o}=0.454 V vs. RHE) calculated based on the standard molar Gibbs energy formation at 298.15 K, FE_{A} is the measured Faradaic efficiency (%) of the product A, and E_{full-cell} is the full-cell voltage measured in MEA system without ohmic loss correction.

SPCC calculation. Under the condition of 298.15 K and 101.3 kPa, SPCC is calculated as follows:

$$Total SPCC = 60 [ s ] \times \sum \frac{Total current [ A ] \times {FE}_{A} \times molar ratio [ \frac{CO}{product A} ]}{\begin{matrix}electrons transferred for every \\ product A molecule \times Faraday ' s Constant\end{matrix}} \div \frac{CO feed rate [ \frac{L}{min} ] \times 1 [ min ]}{\frac{8.314 [ J {mol}^{- 1} {K}^{- 1} ] \times 298.15 [ K ]}{101 \; 300 [ Pa ]}}$$

### DFT Calculations

All Ab initio DFT calculations were performed by employing the projector-augmented wave (PAW) method as implemented in the Vienna Ab initio Simulation Package (VASP). The generalized gradient approximation (GGA) in the parametrization of Perdew-Burke-Ernzerhof (PBE) was implemented to describe the exchange-correlation functional. A plane-wave cutoff of 450 eV and 2×4×1 Γ-centered k-point grids generated by the Monkhorst-Pack scheme were used for all the calculations. A vacuum region of more than 15 Å thickness was included along the perpendicular direction to avoid artificial interactions. The zero damping DFT-D3 method of Grimme was employed to better capture long-range dispersion interactions.

The Ag—Cu(111) and Cu(111) structures were constructed based on our previous study. For the screened Ag—X—Cu (X=Au, Pd, Pt, Ni, Fe, and Ru) systems, one Cu atom on the top layer of a 4-layer (6×3) Ag—Cu(111) supercell was substituted with Au, Pd, Pt, Ni, Fe, and Ru (FIGS. 6 to 13). The position of the substituted atom was determined by the structure with the lowest energy in our benchmark calculations (Supplementary FIG. 14). The number of adsorbed *CO near the coupling sites was determined using the adsorption energy of *CO on Ag—X—Cu relative to Cu (table S2). A monolayer of charged water molecules was included in all initial, transition, and final states of C₁-C₁ and C₁-C₂ coupling above the surface to account for the combined field and solvation effects. Geometries of the initial and final states were optimized by a force-based conjugate gradient algorithm with two upper layers together with the water molecules and adsorbates being allowed to relax, while the atoms in the two lower layers were fixed. The transition states were located using the climbing image nudged elastic band method. The Gibbs free energy (ΔG) for C₁-C₁ and C₁-C₂ coupling were calculated by converting the electronic energy using the equation: ΔG=ΔE+ΔZPE+∫ΔCₚdT−TΔS, where ΔE, ΔZPE, ΔCₚ, and ΔS are the differences in electronic energy, zero-point energy, heat capacity, and entropy, respectively, and T is set to room temperature (298.15K).

### Supplementary Text

### Technoeconomic Analysis

To ascertain the economic potential of the n-propanol production via CORR using renewable electricity, we performed a technoeconomic analysis (TEA) based on the modified model from prior reports. FIG. 30 shows the model we used to calculate the plant-gate levelized cost (unit: US$) for the generation of one tonne of n-propanol and the corresponding quantity of ethanol, ethylene, and H₂ produced in CORR. In the model, we considered the costs for the electrolyzer, catalyst, membrane, installation, balance of plant, input chemicals, electricity, gas and liquid product separation, as well as other operational costs.

Here, as an example, based on the performance (n-propanol FE: 36%, ethanol FE: 19%, ethylene FE: 30%, H₂ FE: 7%, full-cell potential: 2.6 V) achieved on Ag—Ru—Cu catalysts at the operation current density of 300 mA cm⁻² in the A=15 cm² MEA electrolyzer with the CO feed rate of 20.7 mL cm⁻¹, the details for the TEA calculation are listed below.

(1) The cost of the electrolyzer, catalyst, and membrane: Assuming the production capacity of the plant is 100 tonne of n-propanol per day, the total current needed is:

$$Total current needed [ A ] = \frac{\frac{n - PrOH production [ \frac{g}{day} ]}{molecular {weight}_{n - PrOH} [ \frac{g}{mol} ] \times 86400 \frac{s}{day}} \times electrons transferred \times Faraday ' s Constant}{n - PrOH FE [ decimal ]} = \frac{\frac{100 \times \frac{{10}^{6} g}{day}}{\frac{60.09 g}{mol} \times \frac{86400 s}{day}} \times 12 \times 96485 \frac{C}{mol}}{0.36} = 61947331 A$$

Based on the full cell potential (2.6 V) in the experiments, we can get the consumed power as follows:

$$Power Consumed [ W ] = Total current needed [ A ] \times Cell voltage [ V ] = 61947331 A \times 2.6 V = 161063.0606 kW$$

From the DOE H2A analysis for central grid electrolysis, the electrolyzer cost for the stack component is $250.25/kW with a reference current density of 175 mA cm⁻² (18). Thus, the total electrolyzer cost is:

$$Total Electrolyzer Cost ( \$ ) = Power Consumed [ kW ] \times Electrolyzer Cost [ \frac{\$}{kW} ] \times \frac{base current density [ \frac{mA}{{cm}^{2}} ]}{imput current density [ \frac{mA}{{cm}^{2}} ]} = 161063.0606 kW \times 250.25 \frac{\$}{kW} \times \frac{175 \frac{mA}{{cm}^{2}}}{300 \frac{mA}{{cm}^{2}}} = \$23511851 \text{.37}$$

As the total electrolyzer cost above is the one-time cost for the electrolyzer, we need to convert it to a cost for generating one tonne of n-propanol. We assume the lifetime of the electrolyzer is 30 years with no salvage value at the end of the plant's lifetime and a plant capacity factor of 0.9 which means the plant produces n-propanol 328.5 days per year. The electrolyzer cost per tonne of n-propanol is:

$$Electrolyzer cost [ \frac{\$}{tonne n - PrOH} ] = \frac{{CRF}_{electrolyzer} \times Total Electrolyzer Cost [ \$ ]}{Capacity factor \times 365 \frac{day}{year} \times production [ \frac{tonne n - PrOH}{day} ]}$$

Herein, the capital recovery factor (CRF) is based on a discount rate (denoted i; we use 7% for all the CRF calculations) and the material lifetime.

$${CRF}_{electrolyzer} = \frac{{i ( 1 + i )}^{lifetime}}{{( 1 + i )}^{lifetime} - 1}$$

$$Hence , Electrolyzer cost [ \frac{\$}{tonne n - PrOH} ] = \frac{\frac{0.07 {( 1.07 )}^{20}}{{( 1.07 )}^{20} - 1} \times \$23511851 \text{.37}}{0.9 \times 365 \frac{day}{year} \times 100 \frac{ton n - PrO}{day}} = 67. 56 \frac{\$}{tonne n - PrOH}$$

For the catalyst and membrane cost, we assume that their total one-time cost is 5% of the electrolyzer cost with a lifetime of 5 years. We can then reduce to a cost per tonne of n-propanol using the same method as above:

$$Catalyst and membrane cost [ \frac{\$}{tonne n - PrOH} ] = \frac{{CRF}_{catalyst and membrane} \times Total Electrolyzer Cost [ \$ ] \times 5 \%}{Capacity factor \times 365 \frac{day}{year} \times production [ \frac{tonn n - PrOH}{day} ]} = \frac{\frac{0.07 {( 1.07 )}^{5}}{{( 1.07 )}^{5} - 1} \times \$2351185100 \text{.37} \times 0.05}{0.9 \times 365 \frac{day}{year} \times 100 \frac{tonne n - PrO}{day}} = 8.73 \frac{\$}{tonne n - Pr}$$

(2) Electricity cost: By assuming the electricity price is 2 c/kWh (56), the electricity cost per tonne of n-propanol is:

$$Electricity cost [ \frac{\$}{tonne n - PrOH} ] = \frac{Power Consumed [ kW ] \times 24 hours \times electricity price [ \frac{\$}{kWh} ]}{n - PrOH production [ \frac{tonne n - PrOH}{day} ]} = \frac{161063.0 \; 606 kW \times 24 hours \times \frac{0.02 \$}{kWh}}{100 \frac{tonne n - PrOH}{day}} = 773.30 \frac{\$}{ton n - PrOH}$$

(3) Liquid separation cost: Apart from n-propanol, we also consider generated ethanol as a liquid byproduct that can be sold along with n-propanol. Due to the liquid crossover, liquid from the cathode outlet and anolyte from the anode side will be collected for separation. We assume the aqueous solution will be recirculated until the total volume concentration of n-propanol and ethanol reaches 10%. The cost for the liquid separation is calculated using a distillation model. For n-propanol, the distillation model uses a reference cost of $4687910 for a flowrate capacity of 1000 L min⁻¹ with a scaling factor of 0.7 and a distillation operating cost of $17078.9 per day; for ethanol, the distillation model uses a reference cost of $4162240 for a flowrate capacity of 1000 L min⁻¹ with a scaling factor of 0.7 and a distillation operating cost of $10542.8 per day. To simplify the calculation, here we use the parameters of n-propanol above for the calculation of all liquid product separation cost as it has a higher cost of distillation and will give a more conservative estimate.

At 300 mA cm⁻², the ethanol FE is 19%. With 100 tonne of n-propanol produced per day (FE_{n-PrOH}=36%) via CORR, the quantity of ethanol produced per day is calculated according to:

$$\frac{\frac{n - PrOH produaion [ \frac{g}{d a y} ]}{mole cular {weight}_{n - PrOH} [ \frac{g}{m o l} ]} \times electrons transferred}{\frac{Ethanol produaion [ \frac{g}{day} ]}{molecular {weight}_{Ethanol} [ \frac{g}{m o l} ]} \times electrons transferred} = \frac{n - PrOH Fe}{Ethanol FE}$$

We find the ethanol production per day is 60.6 tonne. We can get the flowrate of n-propanol and ethanol according to:

$$Product flowrate [ \frac{L}{min} ] = \frac{Production rate [ \frac{kg product}{day} ] \times 1000 \frac{L}{{m}^{3}}}{Production density [ \frac{kg}{{m}^{3}} ] \times 24 \frac{hour}{day} \times 60 \frac{min}{hour}}$$

The flowrates of n-propanol and ethanol are 86.48 and 53.34 L min⁻¹, respectively. The flowrate of aqueous solution for separation once a product concentration of 10% is achieved is:

$$Aqueous solution flowrate [ \frac{L}{min} ] = \frac{Total product flowrate [ \frac{L}{min} ]}{Product concentration [ decimal ]} = \frac{( 86.48 + 53.34 ) \frac{L}{min}}{0.1} = 1398.2 \frac{L}{min}$$

The distillation capital cost is calculated by scaling the reference cost to the flowrate of aqueous solution.

$$Distillation capital cost [ \$ ] = \$ 4687910 \times {( \frac{Aqueous solution flowrate [ \frac{L}{min} ]}{1000 \frac{L}{min}} )}^{0.7} = \$4687910 \times {( \frac{1398.2 \frac{L}{min}}{1000 \frac{L}{min}} )}^{0.7} = \$5927584.77$$

The distillation capital cost per tonne of n-propanol and the corresponding quantity of ethanol

$$( Denoted Distillation capital cost [ \frac{\$}{tonne n - PrOH} ] )$$

can be written by assuming the distillation facility lifetime is the same as the electrolyzer lifetime:

$$Distillation capital cost [ \frac{\$}{tonne n - PrOH} ] = \frac{{CRF}_{electrolyzer} \times Distillation capital cost [ \$ ]}{Capacity factor \times 365 \frac{day}{y e a r} \times prod uaion [ \frac{tonne n - PrOH}{day} ]} = \frac{\frac{0.07 {( 1.07 )}^{20}}{{( 1.07 )}^{20} - 1} \times \$ 5927584.77}{0.9 \times 365 \frac{d a y}{y e a r} \times 100 \frac{tonne n - PrOH}{day}} = 17.03 \frac{\$}{tonne n - PrOH} l$$

The distillation operational cost per tonne of n-propanol and the corresponding quantity of ethanol

$$( Denoted Distillation operational cost [ \frac{\$}{n - PrOH} ] )$$

is:

$$Distillation operation cost [ \frac{\$}{tonne n - PrOH} ] = \frac{Aqueous solution flowrate [ \frac{L}{min} ]}{1000 \frac{L}{min} \times production [ \frac{tonne n - PrOH}{day} ]} \times 17078.9 \frac{\$}{day} = \frac{1398.2 \frac{L}{min}}{1000 \frac{L}{min} \times 100 \frac{tonne n - PrOH}{day}} \times 17078.9 \frac{\$}{day} = 238.80 \frac{\$}{tonne n - PrOH}$$

The liquid separation cost per tonne of n-propanol and the corresponding quantity of ethanol

$$( Denoted Liquid separation cost [ \frac{\$}{n - PrOH} ] )$$

is:

$$Liquid seperation cost [ \frac{\$}{tonne n - PrOH} ] = Distillation capital cost [ \frac{\$}{tonne n - PrOH} ] + Distillation operational cost [ \frac{\$}{tonne n - PrOH} ] = 17.03 \frac{\$}{tonne n - PrOH} + 238.80 \frac{\$}{tonne n - PrOH} = 255.83 \frac{\$}{tonne n - PrOH}$$

(4) Gas separation cost: We also consider generated ethylene and H₂ as the gas byproducts for sale. The cost for the gas separation from cathode output is calculated using a model which describes the capital and operational cost of a pressure swing adsorption (PSA) system for biogas upgrading. The model uses a reference cost of $1989043 for a flowrate capacity of 1000 m³ h⁻¹ with a scaling factor of 0.7 and an energy consumption of 0.25 kWh m⁻³. The gases in the cathode output include ethylene, H₂, and CO. At 300 mA cm⁻², the FEs of n-propanol, ethylene, and H₂ are 36%, 30%, and 7%, respectively. With 100 tonne of n-propanol produced per day via CORR, the quantity of ethylene and H₂ produced per day is calculated according to:

$$\frac{\frac{n - PrOH produaion [ \frac{g}{d a y} ]}{molecular {weight}_{n - PrOH} [ \frac{g}{m o l} ]} \times electrons transferred}{\frac{{C}_{2} {H}_{4} produaion [ \frac{g}{d a y} ]}{molecular {weight}_{{C}_{2} {H}_{4}} [ \frac{g}{m o l} ]} \times electrons transferred} = \frac{n - PrOH}{{C}_{2} {H}_{4} FE}$$

$$\frac{\frac{n - PrOH produaion [ \frac{g}{d a y} ]}{molecular {weight}_{n - PrOH} [ \frac{g}{mol} ]} \times electrons transferred}{\frac{{H}_{2} p r o d u a i o n [ \frac{g}{d a y} ]}{molecular {weight}_{{H}_{2}} [ \frac{g}{m o l} ]} \times electrons transferred} = \frac{n - PrOH}{{H}_{2} FE}$$

We thus can get ethylene and H₂ production per day of 58.2 and 3.9 tonne, respectively. The output ethylene and H₂ flowrates are:

$$Output {C}_{2} {H}_{4} flowrate [ \frac{{m}^{3}}{h o u r} ] = \frac{58.2 \times 1 {0}^{6} g \times 8.314 J {mol}^{- 1} {K}^{- 1} \times 298.15 K}{28 \frac{g}{m o l} \times 101 \; 300 Pa \times 24 hour} = 2119.3 \frac{{m}^{3}}{h o u r}$$

$$Output {H}_{2} flowrate [ \frac{{m}^{3}}{h o u r} ] = \frac{3.9 \times 1 {0}^{6} g \times 8.3 \; 14 {Jmol}^{- 1} {K}^{- 1} \times 298.15 K}{2 \frac{g}{m o l} \times 101 \; 300 Pa \times 24 hour} = 1988.2 \frac{{m}^{3}}{h o u r}$$

Based on our experimental data (inlet CO gas: 20.7 mL min⁻¹; electrode area: 15 cm²; current density: 300 mA cm⁻²; 298.15 K; 101.3 kPa), by considering the production of n-propanol, ethanol, and ethylene during CORR, the CO single-pass conversion is 70%.

$$Output C O flowrate [ \frac{{m}^{3}}{hour} ] = ( Output {C}_{2} {H}_{4} flowrate [ \frac{{m}^{3}}{hour} ] \times molar ratio [ \frac{C O}{{C}_{2} {H}_{4}} ] + molar ratio [ \frac{C O}{n - P r O H} ] \times \frac{n - P r O H [ \frac{g}{day} ]}{60.09 \frac{g}{mol} \times 24 \frac{hour}{day}} \times \frac{8.3 \; 14 {Jmol}^{- 1} {K}^{- 1} \times 2 98.15 K}{101 \; 300 Pa} + molar ratio [ \frac{C O}{Ethanol} ] \times \frac{Ethanol [ \frac{g}{day} ]}{46 \frac{g}{mol} \times 24 \frac{hour}{day}} \times \frac{8.3 \; 14 {Jmol}^{- 1} {K}^{- 1} \times 2 98.15 K}{101 \; 300 Pa} ) \times \frac{( 100 - single pass conversion [ \% ] )}{single pass conversion [ \% ]} = ( 2119.3 \frac{{m}^{3}}{hour} \times 2 + 3 \times \frac{100000000 \frac{g}{day} \times 8.3 \; 14 {Jmol}^{- 1} {K}^{- 1} \times 2 98.15 K}{60.09 \frac{g}{mol} \times 24 \frac{hour}{day} \times 101 \; 300 Pa} + 2 \times \frac{60600000 \frac{g}{day} \times 8.3 \; 14 {Jmol}^{- 1} {K}^{- 1} \times 2 98.15 K}{46 \frac{g}{mol} \times 24 \frac{hour}{day} \times 1 \; 01300 Pa} ) \times \frac{( 100 - 70 )}{70} = 5149.4 \frac{{m}^{3}}{hour}$$

By summing the flowrate of ethylene, H₂, and CO, the gas flowrate of cathode output is 9256.9 m³ h⁻¹. The PSA capital cost is:

$$PSA capital cost [ \$ ] = \$1989043 \times {( \frac{flowrate [ \frac{{m}^{3}}{hour} ]}{1000 \frac{{m}^{3}}{hour}} )}^{0.7} = \$1989043 \times {( \frac{9256.9 \frac{{m}^{3}}{hour}}{1000 \frac{{m}^{3}}{hour}} )}^{0.7} = \$9444306 \text{.49}$$

By assuming the PSA facility lifetime is the same as the electrolyzer lifetime, the PSA capital cost of the corresponding quantity of ethylene and H₂ per tonne of n-propanol is produced (denoted

$$PSA capital cost [ \frac{\$}{tonne n - P r O H} ]$$

) can be written:

$$PSA capital cost [ \frac{\$}{tonne n - P r O H} ] = \frac{{CRF}_{electrolyzer} \times PSA capital cost [ \$ ]}{Capacity factor \times 365 \frac{day}{year} \times production [ \frac{\$}{tonne n - P r O H} ]} = \frac{\frac{0.07 {( 1.07 )}^{20}}{{( 1.07 )}^{20} - 1} \times \$9444306 \text{.49}}{0.9 \times 365 \frac{day}{year} \times 100 \frac{ton n - P r O H}{day}} = 27.14 \frac{\$}{tonne n - P r O H}$$

The PSA operational cost of the corresponding quantity of ethylene and H₂ per tonne of n-propanol (denoted

$$PSA operational cost [ \frac{\$}{tonne n - P r O H} ]$$

) is:

$$PSA operational cost [ \frac{\$}{tonne n - P r O H} ] = \frac{0.25 \frac{kWh}{{m}^{3}} \times flowrate [ \frac{{m}^{3}}{hour} ] \times 24 \frac{hour}{day} \times electricity price [ \frac{\$}{kWh} ]}{production rate [ \frac{tonne n - P r O H}{day} ]} = \frac{0.25 \frac{kWh}{{m}^{3}} \times 9256.9 \frac{{m}^{3}}{hour} \times 24 \frac{hour}{day} \times 0.02 \frac{\$}{kWh}}{100 \frac{tonne n - P r O H}{day}} = 11.11 \frac{\$}{tonne n - P r O H}$$

The gas separation cost to get the corresponding quantity of ethylene and H₂ per tonne of n-propanol

$$( denoted Gas seperation cost [ \frac{\$}{tonne n - P r O H} ] )$$

is:

$$Gas seperation cost [ \frac{\$}{tonne n - P r O H} ] = PSA capital cost [ \frac{\$}{tonne n - P r O H} ] + PSA operational cost [ \frac{\$}{tonne n - P r O H} ] = 27.14 \frac{\$}{tonne n - P r O H} + 11.11 \frac{\$}{tonne n - P r O H} = 38.25 \frac{\$}{tonne n - P r O H}$$

(5) The total capital costs: By summing the cost for the electrolyzer, catalyst, and membrane, distillation capital cost, and PSA capital cost, we can get the total capital costs of $120.46 per tonne of n-propanol and the corresponding quantity of ethanol, ethylene, and H₂ (denoted

$$Total capital cost [ \frac{\$}{tonne n - P r O H} ]$$

).

(6) Installation cost: We assume a Lang factor of 1 for the calculation of equipment installation cost. The installation cost per tonne of n-propanol and the corresponding quantity of ethanol, ethylene, and H₂

$$( denoted Installation cost [ \frac{\$}{tonne n - P r O H} ] )$$

is:

$$Installation cost [ \frac{\$}{tonne n - P r O H} ] = Lang Factor \times Total capital cost [ \frac{\$}{tonne n - P r O H} ] = 120.46 \frac{\$}{tonne n - P r O H}$$

(7) Balance of plant (BoP): We assume the balance of plant is 50% of the total capital costs. The balance of plant per tonne of n-propanol and the corresponding quantity of ethanol, ethylene, and H₂ (denoted

$$B o P [ \frac{\$}{tonne n - P r O H} ]$$

) is:

$$B o P [ \frac{\$}{tonne n - P r O H} ] = B o P Factor \times Total capital cost [ \frac{\$}{tonne n - P r O H} ] = 50 \% \times 120.46 \frac{\$}{tonne n - P r O H} = 60.23 \frac{\$}{tonne n - P r O H}$$

(8) Input chemicals cost: For the input chemicals cost, we account for the cost from the consumed H₂O, electrolyte, and CO. The water price is estimated as $5 per tonne based on the 2020 water rates (4.0735 $CAD m⁻³) for the city of Toronto, Canada. The cost of water consumed for oxygen reduction reaction per day can be calculated according to:

$$Cost of consumed {H}_{2} O = \frac{\begin{matrix}Total current needed [ A ] \times 86400 \frac{s}{day} \times \\ molecular {weight}_{H 2 O} [ \frac{kg}{mol} ]\end{matrix}}{\begin{matrix}electrons transferred per {H}_{2} O molecule \times \\ Faraday ’ s Constant \times 1000\end{matrix}} \times 5 \frac{\$}{tonne {H}_{2} O} = \frac{619 \; 47331 A \times 86400 \frac{s}{day} \times 0.018 \frac{kg}{mol}}{2 \times 96485 \frac{C}{mol} \times 1000} \times 5 \frac{\$}{tonne {H}_{2} O} = \$ 2496.26$$

The cost of consumed water per tonne of n-propanol and the corresponding quantity of ethanol, ethylene, and H₂

$$( denoted Cost of consumed {H}_{2} O [ \frac{\$}{tonne n - P r O H} ] )$$

is:

$$Cost of consumed {H}_{2} O [ \frac{\$}{tonne n - PrOH} ] = \frac{\$2496 \text{.26}}{100 tonne n - PrOH} = 24.96 \frac{\$}{tonne n - PrOH}$$

The electrolyte is 1 M KOH aqueous solution. We estimate a fixed volume ratio of 100 L electrolyte per m² of electrolyzer based on our lab-scale experiments. The total volume of electrolyte needed is:

$$Volume of electrolyte [ L ] = \frac{Total current needed [ mA ]}{Current density [ \frac{mA}{{cm}^{2}} ] \times {( \frac{100 cm}{1 m} )}^{2}} \times 100 \frac{L}{{m}^{2}} = \frac{61947331000 mA}{300 \frac{mA}{{cm}^{2}} \times {( \frac{100 cm}{1 m} )}^{2}} \times 100 \frac{L}{{m}^{2}} = 2064911.033 L$$

Assuming a price of $800 per tonne for KOH, we can get the total cost of electrolyte including the cost of KOH and the cost of water according to:

$$Cost of electrolyte [ \$ ] = Volume of electrolyte [ L ] \times molecular {weight}_{KOH} [ \frac{kg}{mol} ] \times 1 \frac{mol}{L} \times price of K O H [ \frac{\$}{kg} ] + Volume of electrolyte [ L ] \times water price [ \frac{\$}{kg} ] = 2064911.033 L \times 0.056 \frac{kg}{mol} \times 1 \frac{mol}{L} \times 0.8 \frac{\$}{kg} + 2064911.033 L \times 1 \frac{kg}{L} \times 0.005 \frac{\$}{kg} = \$102832 \text{.57}$$

By assuming an electrolyte lifetime of one year, we calculate a new CRF:

$${CRF}_{electrolyte} = \frac{0.07 {( 1.07 )}^{1}}{{1.07}^{1} - 1} = 1.07$$

For producing 1 tonne of n-propanol and the corresponding quantity of ethanol, ethylene, and H₂, the cost of electrolyte (denoted

$$Cost of electrolyte [ \frac{\$}{tonne n - PrOH} ] )$$

) is:

$$Cost of electrolyte [ \frac{\$}{tonne n - PrOH} ] = \frac{{CRF}_{electrolyte} \times Cost of electrolyte [ \$ ]}{Capacity factor \times 365 \frac{day}{year} \times production [ \frac{tonne n - PrOH}{day} ]} = \frac{1.07 \times \$102832 \text{.57}}{0.9 \times 365 \frac{day}{year} \times 100 \frac{tonne n - PrOH}{day}} = 3.35 \frac{\$}{tonne n - PrOH}$$

By assuming CO price of $300 per tonne, the cost of CO for producing 1 tonne of n-propanol and the corresponding quantity of ethanol, ethylene, and H₂

$$( denoted Cost of CO [ \frac{\$}{tonne n - PrOH} ] )$$

is:

$$Cost of CO [ \frac{\$}{tonne n - PrOH} ] = ( molar ratio [ \frac{CO}{{C}_{2} {H}_{6}} ] \times \frac{{C}_{2} {H}_{4} [ \frac{g}{day} ]}{28 \frac{g}{mol}} + molar ratio [ \frac{CO}{n - PrOH} ] \times \frac{n - PrOH [ \frac{g}{day} ]}{60.09 \frac{g}{mol}} + molar ratio [ \frac{CO}{Ethanol} ] \times \frac{Ethanol [ \frac{g}{day} ]}{46 \frac{g}{mol}} ) \times \frac{28 \frac{g}{mol}}{500 \frac{tonne n - PrOH}{day}} \times price of CO [ \frac{\$}{tonne} ] = ( 2 \times \frac{58200000 \frac{g}{day}}{28 \frac{g}{mol}} + 3 \times \frac{1000000 \frac{g}{day}}{60.09 \frac{g}{mol}} + 2 \times \frac{60600000 \frac{g}{day}}{46 \frac{g}{mol}} ) \times \frac{28 \frac{g}{mol}}{\frac{1000000 g}{tonne} \times 100 \frac{tonne n - PrOH}{day}} \times 300 \frac{\$}{tonne} = 989.89 \frac{\$}{tonne n - PrOH}$$

We can get the input chemicals cost per tonne of n-propanol and the corresponding quantity of ethanol, ethylene, and H₂ (denoted

$$Input chemicals cost [ \frac{\$}{tonne n - PrOH} ] )$$

according to:

$$Input chemical cost [ \frac{\$}{tonne n - PrOH} ] = Cost of consumed {H}_{2} O [ \frac{\$}{tonne n - PrOH} ] + Cost of electrolyte [ \frac{\$}{tonne n - PrOH} ] + Cost of CO [ \frac{\$}{tonne n - PrOH} ] = 24.29 \frac{\$}{tonne n - PrOH} + 3.35 \frac{\$}{tonne n - PrOH} + 989.89 \frac{\$}{tonne n - PrOH} = 1018.2 \frac{\$}{tonne n - PrOH}$$

(9) Other operational costs: Other operational costs (such as labor and maintenance; denoted

$$Other operational costs [ \frac{\$}{tonne n - PrOH} ] )$$

are assumed to be 10% of the electricity cost per tonne of n-propanol and the corresponding quantity of ethanol, ethylene, and H₂:

$$Other operational costs [ \frac{\$}{tonne n - PrOH} ] = Electricity cost [ \frac{\$}{tonne n - PrOH} ] \times 0.1 = 773.1 \frac{\$}{tonne n - PrOH} \times 0.1 = 77.31 \frac{\$}{tonne n - PrOH}$$

(10) The plant-gate levelized cost for producing 1 tonne of n-propanol and the corresponding quantity of ethanol, ethylene, and H₂ (denoted

$$Plant - gate levelized cost [ \frac{\$}{tonne n - PrOH} ] )$$

is:

$$Plant - gate levelized cost [ \frac{\$}{tonne n - PrOH} ] = ( 67.56 + 8.73 + 773.1 + 255.83 + 38.25 + 120.46 + 60.23 + 1018.2 + 77.31 ) \frac{\$}{tonne n - PrOH} = 2419.67 \frac{\$}{tonne n - PrOH}$$

(11) Potential profit: We assume the market prices of n-propanol, ethanol, ethylene, and H₂ per tonne are $1430, $800, $1000, and $1900, respectively. The profit per tonne of n-propanol and the corresponding quantity of ethanol, ethylene, and H₂ can be calculated according to:

$$1 tonne \times 1430 \frac{\$}{tonne} + \frac{60.6}{100} tonne \times 800 \frac{\$}{tonne} + \frac{58.2}{100} tonne \times 1000 \frac{\$}{tonne} + \frac{3.9}{100} tonne \times 1900 \frac{\$}{tonne} - \$2419 \text{.67} = \$2570 \text{.90} - \$2419 \text{.67} = \$151 \text{.23}$$

TABLE S1
n-Propanol electrosynthesis reports having total current density above 100 mA cm⁻².

| | FE_{n-PrOH} | J_{n-PrOH} | Stability | Total | | |
|---|---|---|---|---|---|---|
| Catalysts | (%) | (mA cm⁻²) | (h) | SPCC (%) | Reactions | References |
| Ag—Ru—Cu | 37 ± 3 | 111 ± 9 | 102 | 85 | CORR | This work |
| Cu adparticles | 11 | 26 | N/A | 2 | CORR | Nat. Commun. 9, 4614 (2018) |
| Cavity Cu | 12 | 13 | N/A | <1 | CORR | Nat. Catal. 1, 946-951 (2018) |
| Oxide-derived Cu | 14 | 20 | N/A | 5 | CORR | Nat. Catal. 1, 748-755 (2018) |
| Fragmented Cu | 18 | 33 | N/A | 1 | CORR | Nat. Catal. 2, 251-258 (2019) |
| ClBH Cu | 6 | 26 | N/A | 3 | CO₂RR | Science 367, 661-666 (2020) |
| Cu₂S—Cu—V | 7 | 28 | N/A | N/A | CO₂RR | Nat. Catal. 1, 421-428 (2018) |
| Metal ion cycled Cu | 14 | 15 | N/A | <1 | CO₂RR | Nat. Catal. 1, 111-119 (2018) |

TABLE S2
The adsorption energies (E_{ads}) for the first, second, third,
and fourth *CO near the coupling sites (denoted E_{ad-(1st *CO)}, E_{ad-(2nd *CO)},
E_{ad-(3rd *CO)}, and E_{ad-(4th *CO)}) on screened Ag-X-Cu systems,
as well as the determined geometries of Ag-X-Cu systems with
adsorbed *CO near the coupling sites.

| | E_{ad-(1st *CO)} | E_{ad-(2nd *CO)} | E_{ad-(3rd *CO)} | E_{ad-(4th *CO)} |
|---|---|---|---|---|
| Systems | (eV) | (eV) | (eV) | (eV) |
| Ag-Au-Cu | / | / | / | / |
| Ag-Pd-Cu | −1.43 | −1.08 | / | / |
| Ag-Pt-Cu | −1.75 | −1.01 | / | / |
| Ag-Ni-Cu | −1.99 | −1.23 | / | / |
| Ag-Fe-Cu | −2.73 | −2.24 | −1.51 | / |
| Ag-Ru-Cu | −2.69 | −2.09 | −1.34 | / |

Note:
We assume the C₁-C₁ and C₁-C₂ coupling steps take place on the Cu atoms, and the number of adsorbed *CO near the coupling sites for screened Ag-X-Cu systems is determined by E_{ads}.
The calculated *CO adsorption energy (−0.91 eV) on pure Cu is used as the reference.
If E_{ads} (including E_{ad-(1st *CO)}, E_{ad-(2nd *CO)}, E_{ad-(3rd *CO)}, and E_{ad-(4th *CO)}) is above −0.91 eV, it is more favorable for this *CO to adsorb on/near the X atoms (near the coupling sites) thermodynamically; otherwise, *CO tends to adsorb on Cu atoms only (coupling sites), and we denote this case as “/” in the Table.
Here X in the screened Ag-X-Cu is categorized into three different groups according to the *CO adsorption energies with the reference to Cu, i.e., weak: Au; intermediate: Pd, Pt, and Ni; strong: Fe, and Ru.
Based on the calculations, the number of adsorbed *CO near the coupling sites for Ag-Au-Cu, Ag-Pd-Cu, Ag-Pt-Cu, Ag-Ni-Cu, Ag-Fe-Cu, Ag-Ru-Cu are 0, 2, 2, 2, 3, and 3, respectively.

TABLE S3
The calculated *CO adsorption energies (E_{ads}) on different sites and the most favorable geometries
after optimization for the first, second, third, and fourth *CO adsorbed on Ag-Ru-Cu surface.

| | | Most favorable | | | Most favorable |
|---|---|---|---|---|---|
| Initial sites for the 1^{st} *CO | E_{ads} | geometry after | Initial sites for the 2^{nd} *CO | E_{ads} | geometry after |
| adsorption | (eV) | optimization | adsorption | (eV) | optimization |
| 125_AgCu2FccHollow | −2.69 | 6_RuTop | 125_AgCu2FccHollow | −2.09 | 236_Cu2RuFccHollow + |
| 12_AgCuBridge | −2.69 | | 12_AgCuBridge | −1.06 | 679_Cu2RuHcpHollow |
| 145_AgCu2HcpHollow | −1.04 | | 145_AgCu2HcpHollow | −1.06 | |
| 1_AgTop | −0.34 | | 1_AgTop | −0.47 | |
| 236_Cu2RuFccHollow | −2.69 | | 236_Cu2RuFccHollow | −2.09 | |
| 23_CuCuBridge | −2.69 | | 23_CuCuBridge | −2.09 | |
| 256_Cu2RuHcpHollow | −2.69 | | 256_Cu2RuHcpHollow | −2.09 | |
| 25_CuCuBridge | −2.69 | | 25_CuCuBridge | −2.09 | |
| 26_CuCuBridge | −2.69 | | 26_CuCuBridge | −2.04 | |
| 2_CuTop | −2.69 | | 2_CuTop | −1.06 | |
| 458_Cu3FccHollow | −1.04 | | 458_Cu3FccHollow | −1.07 | |
| 45_CuCuBridge | −1.04 | | 45_CuCuBridge | −1.06 | |
| 478_Cu3HcpHollow | −0.95 | | 478_Cu3HcpHollow | −0.90 | |
| 47_CuCuBridge | −2.69 | | 47_CuCuBridge | −2.09 | |
| 48_CuCuBridge | −1.04 | | 48_CuCuBridge | −1.06 | |
| 6_RuTop | −2.69 | | 78_CuCuBridge | −1.06 | |
| 78_CuCuBridge | −1.04 | | 8_CuTop | −0.94 | |
| 8_CuTop | −0.88 | | | | |

| | | Most favorable | | | Most favorable |
|---|---|---|---|---|---|
| Initial sites for the 3rd *CO | E_{ads} | geometry after | Initial sites for the 4th | E_{ads} | geometry after |
| adsorption | (eV) | optimization | *CO adsorption | (eV) | optimization |
| 125_AgCu2FccHollow | −0.99 | 236_Cu2RuFccHollow + | 125_AgCu2FccHollow | −0.51 | 256_Cu2RuHcpHollow + |
| 12_AgCuBridge | −0.99 | 467_Cu2RuFccHollow + | 12_AgCuBridge | −0.51 | 346_Cu2RuHcpHollow + |
| 145_AgCu2HcpHollow | −0.99 | 569_Cu2RuFccHollow | 145_AgCu2HcpHollow | −0.51 | 679_Cu2RuHcpHollow + |
| 1_AgTop | −0.52 | | 1_AgTop | −0.53 | 8_CuTop |
| 23_CuCuBridge | −1.34 | | 23_CuCuBridge | −0.86 | |
| 2_CuTop | −0.99 | | 2_CuTop | −0.51 | |
| 458_Cu3FccHollow | −0.99 | | 458_Cu3FccHollow | −0.51 | |
| 45_CuCuBridge | −0.99 | | 45_CuCuBridge | −0.51 | |
| 478_Cu3HcpHollow | −0.96 | | 478_Cu3HcpHollow | −0.86 | |
| 48_CuCuBridge | −0.99 | | 48_CuCuBridge | −0.73 | |
| 78_CuCuBridge | −0.99 | | 78_CuCuBridge | −0.72 | |
| 8_CuTop | −0.96 | | 8_CuTop | −0.86 | |

TABLE S4
Gibbs free energies of C₁-C₁ and C₁-C₂ coupling steps on Ag-Ru-Cu
under different number of adsorbed *CO near the coupling sites.

| The number of adsorbed *CO | ΔG (C₁-C₁ coupling) (eV) | ΔG (C₁-C₂ coupling) (eV) |
|---|---|---|
| 0 | 1.23 | 1.74 |
| 1 | 1.30 | 0.91 |
| 2 | 1.00 | 0.17 |
| 3 | 0.51 | −0.63 |

TABLE S5
Product FEs for different electrodes under different applied current densities in CORR.

| | Jₜₒₜₐₗ | FEₙ₋ₚᵣₒₚₐₙₒₗ | FEₑₜₕₐₙₒₗ | FE_{acetate} | FE_{ethylene} | FE_{hydrogen} |
|---|---|---|---|---|---|---|
| Electrodes | (mA cm⁻²) | (%) | (%) | (%) | (%) | (%) |
| Ag—Ru—Cu | 200 | 32.7 ± 2.3 | 15.9 ± 1.3 | 7.9 ± 0.5 | 28.3 ± 0.3 | 11.2 ± 0.1 |
| | 300 | 37.1 ± 2.6 | 16.3 ± 1.3 | 6.6 ± 1.1 | 28.9 ± 0.2 | 10 ± 0.1 |
| | 400 | 28.9 ± 1.0 | 17.6 ± 0.3 | 12.3 ± 0.3 | 30.3 ± 0.1 | 9.8 ± 0.3 |
| | 500 | 30.6 ± 2.3 | 16.7 ± 1.0 | 13.2 ± 2.7 | 31.3 ± 0.9 | 9.1 ± 0.5 |
| | 600 | 25.2 ± 0.9 | 15.1 ± 0.5 | 11.1 ± 0.4 | 37.9 ± 0.2 | 10.9 ± 0.1 |
| Cu | 200 | 20.0 ± 1.9 | 12.8 ± 1.2 | 8.3 ± 2.0 | 33.6 ± 0.5 | 18.8 ± 0.2 |
| | 300 | 20.8 ± 0.3 | 10.8 ± 1.6 | 7.4 ± 0.3 | 36.9 ± 0.8 | 17.4 ± 0.4 |
| | 400 | 20.4 ± 1.7 | 13.5 ± 0.4 | 8.5 ± 0.1 | 36.5 ± 0.3 | 20.9 ± 0.8 |
| | 500 | 20.0 ± 1.5 | 11.1 ± 0.3 | 7.5 ± 0.3 | 35.7 ± 0.8 | 22.6 ± 0.1 |
| | 600 | 18.5 ± 0.3 | 10.4 ± 0.1 | 6.2 ± 0.1 | 35.9 ± 0.9 | 27.9 ± 0.4 |
| Ag—Cu | 200 | 24.7 ± 0.8 | 12.7 ± 0.2 | 9.3 ± 0.5 | 30.3 ± 0.7 | 15.0 ± 0.4 |
| | 300 | 24.3 ± 0.3 | 14.5 ± 1.2 | 9.3 ± 0.4 | 34.8 ± 0.8 | 14.0 ± 0.4 |
| | 400 | 23.4 ± 0.1 | 15.0 ± 0.9 | 10.5 ± 0.2 | 36.9 ± 0.3 | 16.0 ± 0.8 |
| | 500 | 23.0 ± 0.2 | 14.5 ± 0.4 | 9.6 ± 0.5 | 36.2 ± 1.2 | 16.7 ± 0.5 |
| | 600 | 21.3 ± 0.1 | 11.6 ± 0.6 | 9.8 ± 0.2 | 37.4 ± 0.4 | 19.4 ± 0.7 |

Error bars represent the standard deviation of three independent samples.

TABLE S6
Product FEs on Ag-Ru-Cu electrodes having an active geometric
area of 15 cm² under the applied current of 4.5 A in
the scaled MEA electrolyzer. Error bars represent the
standard deviation of three independent samples.

| CO | | | | | |
|---|---|---|---|---|---|
| feed rate | | | | | |
| (mL | FEₙ₋ₚᵣₒₚₐₙₒₗ | FEₑₜₕₐₙₒₗ | FE_{acetate} | FE_{ethylene} | FE_{hydrogen} |
| min⁻¹) | (%) | (%) | (%) | (%) | (%) |
| 20.7 | 35.9 ± 2.5 | 19.0 ± 0.2 | 9.0 ± 0.8 | 29.5 ± 0.6 | 7.1 ± 0.3 |
| 47.0 | 33.0 ± 0.7 | 20.6 ± 0.7 | 8.9 ± 0.6 | 28.5 ± 0.2 | 8.2 ± 0.1 |

TABLE S7
Range of values for sensitivity analysis.

| | Better | Base | Worse |
|---|---|---|---|
| CO price ($/tonne) | 250 | 300 | 350 |
| Electrolyzer cost ($/kW) | 200 | 250.25 | 300 |
| Electricity cost (¢/kWh) | 1 | 2 | 3 |
| n-PrOH FE (%) | 44 | 36 | 10 |
| Current density (mA cm⁻²⁾ | 1000 | 300 | 50 |
| SPCC (%) | 90 | 70 | 5 |
| Full-cell voltage (V) | 2.0 | 2.6 | 5.0 |
| System lifetime (year) | 25 | 20 | 15 |
| Catalyst & membrane lifetime (year) | 10 | 5 | 2.5 |

The following references are also incorporated herein by reference:
1. H.-R. Jhong, S. Ma, P. J. A. Kenis, Electrochemical conversion of CO₂ to useful chemicals: current status, remaining challenges, and future opportunities. Curr. Opin. Chem. Eng. 2, 191-199 (2013).
2. D. Wakerley et al., Bio-inspired hydrophobicity promotes CO₂ reduction on a Cu surface. Nat. Mater. 18, 1222-1227 (2019).
3. H. Wang et al., Synergistic enhancement of electrocatalytic CO₂ reduction to C2 oxygenates at nitrogen-doped nanodiamonds/Cu interface. Nat. Nanotechnol. 15, 131-137 (2020).
4. K. R. Phillips, Y. Katayama, J. Hwang, Y. Shao-Horn, Sulfide-derived copper for electrochemical conversion of CO₂ to formic acid. J. Phys. Chem. Lett. 9, 4407-4412 (2018).
5. R. Ren et al., Molecular electrocatalysts can mediate fast, selective CO₂ reduction in a flow cell. Science 365, 367-369 (2019).
6. J. Gu, C.-S. Hsu, L. Bai, H. M. Chen, X. Hu, Atomically dispersed Fe³⁺ sites catalyze efficient CO₂ electroreduction to CO. Science 364, 1091-1094 (2019).
7. C. Xia et al., Continuous production of pure liquid fuel solutions via electrocatalytic CO₂ reduction using solid-electrolyte devices. Nat. Energy 4, 776-785 (2019).
8. C.-T. Dinh et al., CO₂ electroreduction to ethylene via hydroxide-mediated copper catalysis at an abrupt interface. Science 360, 783-787 (2018).
9. F. Li et al., Molecular tuning of CO₂-to-ethylene conversion. Nature 577, 509-513 (2019).
10. G. L. D. Gregorio et al., Facet-dependent selectivity of Cu catalysts in electrochemical CO₂ reduction at commercially viable current densities. ACS Catal. 10, 4854-4862 (2020).
11. X. Wang et al., Efficient electrically powered CO₂-to-ethanol via suppression of deoxygenation. Nat. Energy 5, 478-486 (2020).
12. M. Jouny, W. Luc, F. Jiao, High-rate electroreduction of carbon monoxide to multi-carbon products. Nat. Catal. 1, 748-755 (2018).
13. M. Jouny, G. S. Hutchings, F. Jiao, Carbon monoxide electroreduction as an emerging platform for carbon utilization. Nat. Catal. 2, 1062-1070 (2019).
14. A. Hauch et al., Recent advances in solid oxide cell technology for electrolysis. Science 370, eaba6118 (2020).
15. R. Kungas, Review-Electrochemical CO₂ reduction for CO production: Comparison of low- and high-temperature electrolysis technologies. J. Electrochem. Soc. 167, 044508 (2020).
16. T. L. Skafte et al., Selective high-temperature CO₂ electrolysis enabled by oxidized carbon intermediates. Nat. Energy 4, 846-855 (2019).
17. J. Klabunde, C. Bischoff, A. J. Papa, “Propanols” in Ullmann's Encyclopedia of Industrial Chemistry (Wiley, 2018).
18. M. Jouny, W. Luc, F. Jiao, General techno-economic analysis of CO₂ electrolysis systems. Ind. Eng. Chem. Res. 57, 2165-2177 (2018).
19. O. S. Bushuyev et al., What should we make with CO₂ and how can we make it? Joule 2, 825-832 (2018).
20. J. Li et al., Copper adparticle enabled selective electrosynthesis of n-propanol. Nat. Commun. 9, 4614 (2018).
21. T.-T. Zhuang et al., Copper nanocavities confine intermediates for efficient electrosynthesis of C₃ alcohol fuels from carbon monoxide. Nat. Catal. 1, 946-951 (2018).
22. Y. Pang et al., Efficient electrocatalytic conversion of carbon monoxide to propanol using fragmented copper. Nat. Catal. 2, 251-258 (2019).
23. F. P. G. de Arquer et al., CO₂ electrolysis to multicarbon products at activities greater than 1 A cm⁻². Science 367, 661-666 (2020).
24. T.-T. Zhuang et al., Steering post-C—C coupling selectivity enables high efficiency electroreduction of carbon dioxide to multi-carbon alcohols. Nat. Catal. 1, 421-428 (2018).
25. K. Jiang et al., Metal ion cycling of Cu foil for selective C—C coupling in electrochemical CO₂ reduction. Nat. Catal. 1, 111-119 (2018).
26. H. Xiao, T. Cheng, W. A. Goddard, Atomistic mechanisms underlying selectivities in C₁ and C₂ products from electrochemical reduction of CO on Cu (111). J. Am. Chem. Soc. 139, 130-136 (2016).
27. K. P. Kuhl, E. R. Cave, D. N. Abram, T. F. Jaramillo, New insights into the electrochemical reduction of carbon dioxide on metallic copper surfaces. Energy Environ. Sci. 5, 7050-7059 (2012).
28. X. Wang et al., Efficient upgrading of CO to C₃ fuel using asymmetric C—C coupling active sites. Nat. Commun. 10, 5186 (2019).
29. J. H. Montoya, C. Shi, K. Chan, J. K. Norskov, Theoretical insights into a CO dimerization mechanism in CO₂. J. Phys. Chem. Lett. 6, 2032-2037 (2015).
30. H. Xiao, W. A. Goddard, T. Cheng, Y. Liu, Cu metal embedded in oxidized matrix catalyst to promote CO₂ activation and CO dimerization for electrochemical reduction of CO₂. Proc. Natl Acad. Sci. USA 114, 6685-6688 (2017).
31. Y. Lum, T. Cheng, W. A. Goddard, J. W. Ager, Electrochemical CO reduction builds solvent water into oxygenate products. J. Am. Chem. Soc. 140, 9337-9340 (2018).
32. J. Hussain, H. Jonsson, E. Skulason, Calculations of product selectivity in electrochemical CO₂ reduction. ACS Catal. 8, 5240-5249 (2018).
33. J. D. Goodpaster, A. T. Bell, M. Head-Gordon, Identification of possible pathways for C—C bond formation during electrochemical reduction of CO₂: New theoretical insights from an improved electrochemical model. J. Phys. Chem. Lett. 7, 1471-1477 (2016).
34. C. M. Cobley, Y. Xia, Engineering the properties of metal nanostructures via galvanic replacement reactions. Mater. Sci. Eng. R: Rep. 70, 44-62 (2010).
35. E. L. Clark, C. Hahn, T. F. Jaramillo, A. T. Bell, Electrochemical CO₂ reduction over compressively strained CuAg surface alloys with enhanced multi-carbon oxygenate selectivity. J. Am. Chem. Soc. 139, 15848-15857 (2017).
36. L. Han, P. Wang, H. Liu, Q. Tan, J. Yang, Balancing the galvanic replacement and reduction kinetics for the general formation of bimetallic CuM (M=Ru, Rh, Pd, Os, Ir, and Pt) hollow nanostructures. J. Mater. Chem. A 4, 18354-18365 (2016)
37. C. M. Gabardo et al., Continuous carbon dioxide electroreduction to concentrated multi-carbon products using a membrane electrode assembly. Joule 3, 2777-2791 (2019).
38. H. Mistry et al., Highly selective plasma-activated copper catalysts for carbon dioxide reduction to ethylene. Nat. Commun. 7, 12123 (2016).
39. Y. Zhou et al., Dopant-induced electron localization drives CO₂ reduction to C₂ hydrocarbons. Nat. Chem. 19, 974-980 (2018).
40. T. T. H. Hoang et al., Nanoporous copper-silver alloys by additive-controlled electrodeposition for the selective electroreduction of CO₂ to ethylene and ethanol. J. Am. Chem. Soc. 140, 5791-5797 (2018).
41. C. M. Gunathunge et al., Spectroscopic observation of reversible surface reconstruction of copper electrodes under CO₂ reduction. J. Phys. Chem. C 121, 12337-12344 (2017).
42. N. Sheppard, T. T. Nguyen, in Advances in Infrared and Raman Spectroscopy, R. J. H. Clark, R. E. Hester, Eds (Heyden, London, 1978), vol. 5.
43. C. M. Gunathunge, V. J. Ovalle, Y. Li, M. J. Janik, M. M. Waegele, Existence of an electrochemically inert CO population on Cu electrodes in alkaline pH. ACS Catal. 8, 7507-7516 (2018).
44. A. Fielicke, P. Gruene, G. Meijer, D. M. Rayner, The adsorption of CO on transition metal clusters: A case study of cluster surface chemistry. Sur. Sci. 603, 1427-1433 (2009).
45. R. B. Sandberg, J. H. Montoya, K. Chan, J. K. Norskov, CO—CO coupling on Cu facets: Coverage, strain and field effects. Sur. Sci. 654, 56-62 (2016).
46. H.-R. Jhong, S. Ma, P. J. A. Kenis, Electrochemical conversion of CO₂ to useful chemicals: current status, remaining challenges, and future opportunities. Curr. Opin. Chem. Eng. 2, 191-199 (2013).
47. D. Wakerley et al., Bio-inspired hydrophobicity promotes CO₂ reduction on a Cu surface. Nat. Mater. 18, 1222-1227 (2019).
48. H. Wang et al., Synergistic enhancement of electrocatalytic CO₂ reduction to C₂ oxygenates at nitrogen-doped nanodiamonds/Cu interface. Nat. Nanotechnol. 15, 131-137 (2020).
49. K. R. Phillips, Y. Katayama, J. Hwang, Y. Shao-Horn, Sulfide-derived copper for electrochemical conversion of CO₂ to formic acid. J. Phys. Chem. Lett. 9, 4407-4412 (2018).
50. R. Ren et al., Molecular electrocatalysts can mediate fast, selective CO₂ reduction in a flow cell. Science 365, 367-369 (2019).
51. J. Gu, C.-S. Hsu, L. Bai, H. M. Chen, X. Hu, Atomically dispersed Fe³⁺ sites catalyze efficient CO₂ electroreduction to CO. Science 364, 1091-1094 (2019).
52. C. Xia et al., Continuous production of pure liquid fuel solutions via electrocatalytic CO₂ reduction using solid-electrolyte devices. Nat. Energy 4, 776-785 (2019).
53. C.-T. Dinh et al., CO₂ electroreduction to ethylene via hydroxide-mediated copper catalysis at an abrupt interface. Science 360, 783-787 (2018).
54. F. Li et al., Molecular tuning of CO₂-to-ethylene conversion. Nature 577, 509-513 (2019).
55. G. L. D. Gregorio et al., Facet-dependent selectivity of Cu catalysts in electrochemical CO₂ reduction at commercially viable current densities. ACS Catal. 10, 4854-4862 (2020).
56. X. Wang et al., Efficient electrically powered CO₂-to-ethanol via suppression of deoxygenation. Nat. Energy 5, 478-486 (2020).
57. M. Jouny, W. Luc, F. Jiao, High-rate electroreduction of carbon monoxide to multi-carbon products. Nat. Catal. 1, 748-755 (2018).
58. M. Jouny, G. S. Hutchings, F. Jiao, Carbon monoxide electroreduction as an emerging platform for carbon utilization. Nat. Catal. 2, 1062-1070 (2019).
59. A. Hauch et al., Recent advances in solid oxide cell technology for electrolysis. Science 370, eaba6118 (2020).
60. R. Kungas, Review-Electrochemical CO₂ reduction for CO production: Comparison of low- and high-temperature electrolysis technologies. J. Electrochem. Soc. 167, 044508 (2020).
61. T. L. Skafte et al., Selective high-temperature CO₂ electrolysis enabled by oxidized carbon intermediates. Nat. Energy 4, 846-855 (2019).
62. J. Klabunde, C. Bischoff, A. J. Papa, “Propanols” in Ullmann's Encyclopedia of Industrial Chemistry (Wiley, 2018).
63. M. Jouny, W. Luc, F. Jiao, General techno-economic analysis of CO₂ electrolysis systems. Ind. Eng. Chem. Res. 57, 2165-2177 (2018).
64. O. S. Bushuyev et al., What should we make with CO₂ and how can we make it? Joule 2, 825-832 (2018).
65. J. Li et al., Copper adparticle enabled selective electrosynthesis of n-propanol. Nat. Commun. 9, 4614 (2018).
66. T.-T. Zhuang et al., Copper nanocavities confine intermediates for efficient electrosynthesis of C₃ alcohol fuels from carbon monoxide. Nat. Catal. 1, 946-951 (2018).
67. Y. Pang et al., Efficient electrocatalytic conversion of carbon monoxide to propanol using fragmented copper. Nat. Catal. 2, 251-258 (2019).
68. F. P. G. de Arquer et al., CO₂ electrolysis to multicarbon products at activities greater than 1 A cm⁻². Science 367, 661-666 (2020).
69. T.-T. Zhuang et al., Steering post-C—C coupling selectivity enables high efficiency electroreduction of carbon dioxide to multi-carbon alcohols. Nat. Catal. 1, 421-428 (2018).
70. K. Jiang et al., Metal ion cycling of Cu foil for selective C—C coupling in electrochemical CO₂ reduction. Nat. Catal. 1, 111-119 (2018).
71. H. Xiao, T. Cheng, W. A. Goddard, Atomistic mechanisms underlying selectivities in C₁ and C₂ products from electrochemical reduction of CO on Cu (111). J. Am. Chem. Soc. 139, 130-136 (2016).
72. K. P. Kuhl, E. R. Cave, D. N. Abram, T. F. Jaramillo, New insights into the electrochemical reduction of carbon dioxide on metallic copper surfaces. Energy Environ. Sci. 5, 7050-7059 (2012).
73. X. Wang et al., Efficient upgrading of CO to C₃ fuel using asymmetric C—C coupling active sites. Nat. Commun. 10, 5186 (2019).
74. J. H. Montoya, C. Shi, K. Chan, J. K. Norskov, Theoretical insights into a CO dimerization mechanism in CO₂. J. Phys. Chem. Lett. 6, 2032-2037 (2015).
75. H. Xiao, W. A. Goddard, T. Cheng, Y. Liu, Cu metal embedded in oxidized matrix catalyst to promote CO₂ activation and CO dimerization for electrochemical reduction of CO₂. Proc. Natl Acad. Sci. USA 114, 6685-6688 (2017).
76. Y. Lum, T. Cheng, W. A. Goddard, J. W. Ager, Electrochemical CO reduction builds solvent water into oxygenate products. J. Am. Chem. Soc. 140, 9337-9340 (2018).
77. J. Hussain, H. Jonsson, E. Skulason, Calculations of product selectivity in electrochemical CO₂ reduction. ACS Catal. 8, 5240-5249 (2018).
78. J. D. Goodpaster, A. T. Bell, M. Head-Gordon, Identification of possible pathways for C—C bond formation during electrochemical reduction of CO₂: New theoretical insights from an improved electrochemical model. J. Phys. Chem. Lett. 7, 1471-1477 (2016).
79. C. M. Cobley, Y. Xia, Engineering the properties of metal nanostructures via galvanic replacement reactions. Mater. Sci. Eng. R: Rep. 70, 44-62 (2010).
80. E. L. Clark, C. Hahn, T. F. Jaramillo, A. T. Bell, Electrochemical CO₂ reduction over compressively strained CuAg surface alloys with enhanced multi-carbon oxygenate selectivity. J. Am. Chem. Soc. 139, 15848-15857 (2017).
81. L. Han, P. Wang, H. Liu, Q. Tan, J. Yang, Balancing the galvanic replacement and reduction kinetics for the general formation of bimetallic CuM (M=Ru, Rh, Pd, Os, Ir, and Pt) hollow nanostructures. J. Mater. Chem. A 4, 18354-18365 (2016).
82. C. M. Gabardo et al., Continuous carbon dioxide electroreduction to concentrated multi-carbon products using a membrane electrode assembly. Joule 3, 2777-2791 (2019).
83. H. Mistry et al., Highly selective plasma-activated copper catalysts for carbon dioxide reduction to ethylene. Nat. Commun. 7, 12123 (2016).
84. Y. Zhou et al., Dopant-induced electron localization drives CO₂ reduction to C₂ hydrocarbons. Nat. Chem. 19, 974-980 (2018).
85. T. T. H. Hoang et al., Nanoporous copper-silver alloys by additive-controlled electrodeposition for the selective electroreduction of CO₂ to ethylene and ethanol. J. Am. Chem. Soc. 140, 5791-5797 (2018).
86. C. M. Gunathunge et al., Spectroscopic observation of reversible surface reconstruction of copper electrodes under CO₂ reduction. J. Phys. Chem. C 121, 12337-12344 (2017).
87. N. Sheppard, T. T. Nguyen, in Advances in Infrared and Raman Spectroscopy, R. J. H. Clark, R. E. Hester, Eds (Heyden, London, 1978), vol. 5.
88. C. M. Gunathunge, V. J. Ovalle, Y. Li, M. J. Janik, M. M. Waegele, Existence of an electrochemically inert CO population on Cu electrodes in alkaline pH. ACS Catal. 8, 7507-7516 (2018).
89. A. Fielicke, P. Gruene, G. Meijer, D. M. Rayner, The adsorption of CO on transition metal clusters: A case study of cluster surface chemistry. Sur. Sci. 603, 1427-1433 (2009).
90. R. B. Sandberg, J. H. Montoya, K. Chan, J. K. Norskov, CO—CO coupling on Cu facets: Coverage, strain and field effects. Sur. Sci. 654, 56-62 (2016).
91. W. Luc, J. Rosen, F. Jiao, An Ir-based anode for a practical CO₂ electrolyzer. Catal. Today 288, 79-84 (2017).
92. B. Ravel, M. Newville, ATHENA, ARTEMIS, HEPHAESTUS: data analysis for X-ray absorption spectroscopy using IFEFFIT. J. Synchrotron Radiat. 12, 537-541 (2005).
93. G. Kresse, J. Furthmuller, Efficient iterative schemes for ab initio total-energy calculations using a plane-wave basis set. Phys. Rev. B 54, 11169-11186 (1996).
94. G. Kresse, J. Furthmuller, Efficiency of ab-initio total energy calculations for metals and semiconductors using a plane-wave basis set. Comp. Mater. Sci. 6, 15-50 (1996).
95. G. Kresse, J. Hafner, Ab initio molecular-dynamics simulation of the liquid-metal amorphous-semiconductor transition in germanium. Phys. Rev. B 49, 14251-14269 (1994).
96. G. Kresse, J. Hafner, Ab initio molecular dynamics for liquid metals. Phys. Rev. B 47, 558-561 (1993).
97. J. P. Perdew, K. Burke, M. Ernzerhof, Generalized gradient approximation made simple. Phys. Rev. Lett. 77, 3865-3868 (1996).
98. H. J. Monkhorst, J. D. Pack, Special points for Brillouin-zone integrations. Phys. Rev. B 13, 5188-5192 (1976).
99. J. H. Montoya, C. Shi, K. Chan, J. K. Norskov, Theoretical insights into a CO dimerization mechanism in CO₂ electroreduction. J. Phys. Chem. Lett. 6, 2032-2037 (2015).
100. S. Grimme, J. Antony, S. Ehrlich, H. Krieg, A consistent and accurate ab initio parametrization of density functional dispersion correction (DFT-D) for the 94 elements H-Pu. J. Chem. Phys. 132, 154104 (2010).
101. P. De Luna et al., What would it take for renewably powered electrosynthesis to displace petrochemical processes? Science 364, eaav3506 (2019).
102. A. Paturska, M. Repele, G. Bazbauers, Economic assessment of biomethane supply system based on natural gas infrastructure. Energy Procedia 72, 71-78 (2015).
103. City of Toronto, “2019 Water Rates & Fees” (https://www.toronto.ca/servicespayments/property-taxes-utilities/utility-bilI/water-rates-and-fees-copy/2020-water-ratesfees/).
104. “China CN: Market Price: Monthly Avg: Inorganic Chemical Material: Potassium Hydroxide 92%” (https://www.ceicdata.com/en/china/china-petroleum--chemical-industry-association-petrochemical-price-inorganic-chemical-material/cn-market-price-monthly-avg-inorganic-chemical-material-potassium- hydroxide-92)
105. W. R. Leow et al., Chloride-mediated selective electrosynthesis of ethylene and propylene oxides at high current density. Science 368, 1228-1233 (2020).
106. “Echemi Market Analysis” (www.echemi.com/productslnformation/pd2017011116051595-1-propanol.html).
107. PCT application No. PCT/CA2020/051597 (Wang & Sargent) entitled UPGRADING OF CO TO C3 PRODUCTS USING MULTI-METALLIC ELECTROREDUCTION CATALYSTS WITH ASSYMETRIC ACTIVE SITES.

In addition, it is noted that any of the particular values disclosed herein can be considered as being ±10% for disclosure purposes. Thus, for example, if a concentration value of 1 g/L is mentioned herein, it should be considered that the range 0.9 to 1.1 g/L is disclosed. It is also noted that one or more features (e.g., values, ranges, pieces of equipment or features thereof, operating conditions, sizes, etc.) disclosed herein can be combined with any other combination of features. For example, if a size of 100 nm is disclosed for a certain nanoparticle herein, it should be noted that the multi-metallic nanoparticle catalyst material disclosed herein can be, in an optional embodiment, of this size (i.e., 100 nm in addition to within the range of 90 nm to 110 nm as per the ±10% disclosure). Various other combinations of features are also possible and should be considered as being disclosed herein.

1. A method of manufacturing a co-doped multi-metallic electrocatalyst for use in electroreduction, the method comprising:
providing a copper (Cu) material comprising Cu nanoparticles;
in a first doping stage, doping the Cu material with a first-stage dopant metal selected from Ruthenium (Ru), Rhodium (Rh), Iridium (Ir), Palladium (Pd), Gold (Au) and Platinum (Pt) to produce a doped Cu material; and
in a second doping stage, doping the doped Cu material with silver (Ag) to produce the co-doped multi-metallic material.

2. The method of claim 1, wherein the first doping stage comprises first-stage galvanic replacement of Cu atoms with atoms of the first-stage dopant metal.

3. The method of claim 2, wherein the first-stage galvanic replacement comprises contacting the Cu material with a first-stage doping solution comprising cations of the first-stage dopant metal.

4. The method of claim 3, wherein the first-stage doping solution comprises a chloride salt of the first-stage dopant metal.

5. The method of claim 3, wherein the first-stage doping solution comprises a nitrate salt of the first-stage dopant metal.

6. The method of claim 1, wherein the second doping stage comprises second-stage galvanic replacement of Cu atoms with Ag atoms.

7. The method of claim 6, wherein the second-stage galvanic replacement comprises contacting the doped Cu material with a second-stage doping solution comprising Ag cations.

8. (canceled)

9. The method of claim 1, wherein the first-stage dopant comprises Ru.

10. (canceled)

11. (canceled)

12. (canceled)

13. (canceled)

14. (canceled)

15. The method of claim 1, wherein the co-doped multi-metallic material is a tri-metallic material.

16. The method of claim 1, wherein the co-doped multi-metallic material has a first-stage dopant concentration between 0.5 wt % and 10 wt %, measured with XPS.

17. (canceled)

18. The method of claim 1, wherein the co-doped multi-metallic material has a Ag concentration between 1 wt % and 10 wt %, measured with XPS.

19. (canceled)

20. (canceled)

21. The method of claim 1, wherein the co-doped multi-metallic material has a first-stage dopant to Ag ratio between 1:2 and 1:7, measured with XPS.

22. (canceled)

23. The method of claim 1, wherein the Cu nanoparticles are deposited onto a gas diffusion substrate prior to the first and second doping stages, wherein the Cu nanoparticles are deposited in a Cu layer on a side of the gas diffusion substrate, and wherein the Cu layer has a thickness between 30 microns and 100 microns.

24. (canceled)

25. (canceled)

26. The method of claim 1, wherein the co-doped multi-metallic material has a morphology that is the same as that of the Cu nanoparticles, and wherein the morphology is generally spheroid in shape, determined from SEM or TM imaging.

27. (canceled)

28. The method of claim 1, wherein the co-doped multi-metallic material is in the form of nanoparticles, and wherein the nanoparticles of the co-doped multi-metallic material have an average size between about 20 nm and about 200 nm, measured based on SEM or TEM imaging.

29. (canceled)

30. (canceled)

31. The method of claim 1, further comprising:
depositing the Cu nanoparticles onto a substrate to form a coated substrate;
immersing the coated substrate in a first-stage doping solution comprising the first-stage dopant metal in cationic form to induce galvanic replacement and form a first-stage coated substrate comprising the doped Cu material;
removing the first-stage coated substrate from the first-stage doping solution;
immersing the first-stage coated substrate in a second-stage doping solution comprising Ag in cationic form to induce galvanic replacement and form a second-stage coated substrate comprising the co-doped multi-metallic material; and
removing the second-stage coated substrate from the second-stage doping solution.

32. The method of claim 31, wherein the first-stage doping solution has a first-stage dopant metal concentration between 1 micromole/L and 10 millimole/L, wherein the first-stage doping solution has a temperature between 25 degrees Celsius and 80 degree Celsius; wherein the method further comprises, after removing the first-stage coated substrate from the first-stage doping solution, washing the coated substrate from the first-stage doping solution with deionized water, and drying the washed coated substrate with an inert gas.

33. (canceled)

34. (canceled)

35. (canceled)

36. (canceled)

37. The method of claim 32, wherein the second-stage doping solution has a second-stage dopant metal concentration between 1 micromole/L and 10 millimole/L, wherein the second-stage doping solution has a second-stage temperature between 25 degrees Celsius and 80 degree Celsius, wherein the method further comprises washing the second-stage coated substrate with deionized water, and drying the washed second-stage coated substrate with an second-stage inert gas.

38-66. (canceled)

67. A process for electrochemical production of propanol from a carbon-containing gas selected from CO and CO₂, comprising:
contacting the carbon-containing gas and an electrolyte with an electrode comprising the co-doped multi-metallic electrocatalyst as manufactured by the method as defined in claim 1, such that the carbon-containing gas contacts the electrocatalyst;
applying a voltage to provide a current density to cause the carbon-containing gas contacting the electrocatalyst to be electrochemically converted into propanol; and
recovering the propanol.

68. (canceled)

69. A co-doped multi-metallic electrocatalyst for electroreduction of CO or CO₂ to produce n-propanol, comprising copper (Cu) co-doped with silver (Ag) and a secondary dopant selected from Ruthenium (Ru), Rhodium (Rh), Iridium (Ir), Palladium (Pd), Gold (Au) and Platinum (Pt), wherein the co-doped multi-metallic electrocatalyst has a secondary dopant concentration between 0.5 wt % and 10 wt % measured with XPS, a Ag concentration between 1 wt % and 10 wt % measured with XPS, and a secondary dopant to Ag ratio between 1:1 and 1:10 measured with XPS.

70.-76. (canceled)
